(19) United States
(12) Patent Application Publication
(10) Pub. No.: US 2025/0129265 A1
(43) Pub. Date: Apr. 24, 2025

(54) NITRIC OXIDE RELEASING POLYSILOXANES AND METHODS FOR MAKING AND USING THE SAME

(71) Applicant: University of Georgia Research Foundation, Inc., Athens, GA (US)

(72) Inventors: Elizabeth J. Brisbois, Athens, GA (US); Yun Qian, Athens, GA (US)

(21) Appl. No.: 18/693,198

(22) PCT Filed: Sep. 19, 2022
(86) PCT No.: PCT/US2022/076627

§ 371 Date: Mar. 19, 2024

Related U.S. Application Data
(60) Provisional application No. 63/246,144, filed on Sep. 20, 2021.

(51) Int. Cl. C09D 183/08; A01N 55/00; A01P 1/00; A61L 2/232; A61L 101/46; C08G 77/26; C08G 77/28; C08G 77/388; C08G 77/392; C09D 5/14

(52) U.S. Cl. CPC C09D 183/08; A01N 55/00; A01P 1/00; A61L 2/232; C08G 77/26; C08G 77/28; C08G 77/388; C08G 77/392; C09D 5/14; A61L 2101/46

(57) ABSTRACT

Described herein are nitric oxide-releasing compositions. In one aspect, the nitric oxide releasing polysiloxane comprises a polysiloxane backbone with one or more nitric oxide releasing moieties pendant to the polysiloxane backbone. The nitric oxide-releasing compositions possess antibacterial properties with respect to both Gram-positive and Gram-negative bacteria. The ease of synthesis and excellent antibacterial effects against both Gram-positive and Gram-negative bacteria without resistance and serious leaching concerns makes the nitric oxide-releasing compositions useful as lubricants for medical devices and other articles where it is desirable to reduce or prevent bacterial infection.

### CROSS-REFERENCE TO RELATED APPLICATIONS

This application claims the benefit of and priority to co-pending U.S. Provisional Patent Application No. 63/246,144, filed on Sep. 20, 2021, the contents of which are incorporated by reference herein in their entireties.

### BACKGROUND

Bacterial infection and biofilm formation are prevalent in daily life, and they are critical reasons for many diseases and indwelling medical device failures. Many bacteria can cause infections, such as Gram-positive Staphylococcus aureus (S. aureus), Staphylococcus epidermidis (S. epidermidis), and Streptococcus pyogenes (S. pyogenes), and Gram-negative Pseudomonas aeruginosa (P. aeruginosa) and Escherichia coli (E. coli) [1-3]. When these pathogens aggregate and irreversibly attach to surfaces, they form multicellular communities in extracellular polymeric substances known as biofilms [4]. Biofilms constitute a protective environment to allow bacteria to grow under hostile conditions. Compared to planktonic cells, bacterial cells in biofilms are much more difficult to kill and can lead to persistent and chronic infections [5]. Bacterial infections and biofilms also cause severe diseases with high mortality and morbidity. For example, in 2019, World Health Organization (WHO) reported that infections were associated with more than 30 million deaths worldwide. Five of the top ten causes of these deaths were directly or indirectly associated with bacterial infection or biofilms [6]. Additionally, ˜ 80% of the infections in humans are related directly to biofilm formation, and about 26% of all health-care-associated infections are device-related infections [1]. Failures of indwelling medical devices like intravascular catheters [7, 8], urinary catheters [3], endotracheal tubes [9], tracheostomies [10], enteral feeding tubes [11] and wound drains [12] are widely reported [1, 4, 13]. These infections and biofilms not only bring risks to the uses of devices and implants, but also impact the disease treatments as well as the life quality of patients. For example, 1.6 million T1D diabetic patients require lifetime exogenous insulin, and continuous subcutaneous insulin infusion (CSII) therapy is vital for these patients [14]. However, the skin complications, including infection, inflammation and encapsulation at the insulin cannula infusion site, lead to 65% of infusion set failure after 7 days and result in the discontinuation of CSII therapy [15-17]. Because of the large number of infection cases, fighting bacterial infection and biofilm formation are critical in both disease treatment and application of medical devices.

Although antibiotics have been used extensively to treat bacterial infections and biofilms, more and more concerns about the antibiotic resistance arise, resulting in the uprising demanding for new therapies that kill bacteria while not inducing resistance [18]. Nitric oxide (NO) is a gasotransmitter that is produced endogenously and plays vital roles in regulating various physiological pathways. NO displays therapeutic effects such as antithrombosis, antiplatelet, anti-inflammation, angiogenesis, vascular relaxation, as well as antiviral and antibacterial properties [19-23]. The antibacterial properties of NO are based on the nitrosative and oxidative stress which lead to direct modification of membrane proteins, lipid peroxidation and DNA cleavage [24]. It is noteworthy that NO was reported to eliminate various bacterial strains without increase of bacterial resistance [25]. Pathogens like S. aureus, methicillin-resistant S. aureus (MRSA), S. epidermidis, E. coli and P. aeruginosa did not show resistance after NO exposure. Repeated NO exposure of 20 passages of bacteria did not exhibit any increase of minimum inhibitory concentration (MIC), either. To take advantage of the excellent antibacterial effects of NO, and to stabilize and control NO delivery, many NO-releasing materials have been studied and reported including polysaccharides [26], polyurethane [27], polyvinyl chloride [28], polylactic acid-co-glycolic acid (PLGA) [29], polycarbonate-urethane [30], silicone-polyurethane [31], silicone [32], etc. Among these polymeric materials, silicones (polysiloxane) are widely accepted polymers with long history in biomedical and bioengineering [33]. Due to their immunological inert nature, good mechanical properties, thermal stability, permeable to gases, and biocompatibility of silicones, silicone rubbers (solid silicone materials) have been used for contact lens, cannulas, catheters, grafts and implants, scaffold, wound dressing, while silicone oils (liquid silicones) have been used for cosmetic (e.g. hair care, skin care, etc.) applications and biomedical lubricant applications [34-38].

Silicone materials are widely used in biomedical and bioengineering fields because of their safety, stability and outstanding mechanical properties; therefore, silicones have been used to deliver NO for antibacterial purposes, as well as extend the applications of NO in biomedical and bioengineering area. To date, several studies have utilized various methods to incorporate NO release properties into silicone materials that exhibited antimicrobial and antithrombotic effects. In these studies, SNAP was immobilized to crosslinked silicones by chemical reactions and impregnated or blended into commercial silicone rubbers [40, 41]. NONOate was also formed in situ in a PDMS-based polyurethane by the reactions between polyethyleneimine and NO gas. It is noteworthy that liquid silicone oils have been used to create slippery liquid-infused porous surfaces (SLIPs) by soaking materials with porous surfaces in silicone oils [42], and the resulting SLIPs surfaces are antibacterial and antifouling [43, 44]. Therefore, liquid silicone oils were also utilized to fabricate Liquid-infused NO releasing (LINORel) silicone materials via SLIPS method. LINORel was obtained by impregnating SNAP into silicone rubber first, then infusing a thin layer of silicone oil on the surface, which exhibited both antibacterial and antifouling properties [32, 45]. Materials in these studies showed great antibacterial results, nevertheless, they still have some shortcomings. For example, the reported SNAP-silicone crosslinked polymers went through a complicated synthesis and the crosslinking happened spontaneously, so the manufacturing step (film casting) must be done immediately after the synthesis and this material is hard to use outside the lab environment. Impregnation or blending of SNAP into polymers are more practical as the treatment step only needs encapsulation of SNAP, which does not require synthesis step and is easy to do. However, the SNAP leaching issue is a big concern for material safety because SNAP is a small molecule which tends to diffuse out. The LINORel treatment of silicone oil improved the antibacterial and antifouling performance, but the leaching issue is not completely solved, and this method requires both SNAP impregnation and the SLIPS treatment. Therefore, explore a new solution that provide NO release without leaching issue, and making antibacterial surfaces with easy steps could be very beneficial.

There remains a need for improved materials, articles, and methods that overcome the aforementioned deficiencies.

### SUMMARY

Described herein are nitric oxide-releasing compositions. In one aspect, the nitric oxide releasing polysiloxane comprises a polysiloxane backbone with one or more nitric oxide releasing moieties pendant to the polysiloxane backbone. The nitric oxide-releasing compositions possess antibacterial properties with respect to both Gram-positive and Gram-negative bacteria. The ease of synthesis and excellent antibacterial effects against both Gram-positive and Gram-negative bacteria without resistance and serious leaching concerns makes the nitric oxide-releasing compositions useful as lubricants for medical devices and other articles where it is desirable to reduce or prevent bacterial infection.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be readily appreciated upon review of the detailed description of its various embodiments, described below, when taken in conjunction with the accompanying drawings.

FIGS. 1A-1B show (A) the synthesis scheme of NAP-Si oil and SNAP-Si oil and (B) the concentrations of functional groups on silicone oils as measured by UV-vis spectroscopy. [NH₂] of NH₂—Si oil was determined by ninhydrin assay, [HS] of NAP-Si was calculated by subtracting remaining [NH₂] from the initial [NH₂] of NH₂—Si, and [SNAP] of SNAP-Si oil was measured by SNAP calibrating curve in THF. (n=3 and error bars represent standard deviations).

FIGS. 2A-2B show (A) FT-IR of NH₂—Si, NAP-Si and SNAP-Si and (B) ¹³C NMR of SNAP-Si oil using CDCl₃. Chemical shift at 53.57 ppm represented the characteristic carbon linked to SNO group.

FIG. 3 shows the stability of SNAP-Si measured at three different storage conditions-20° C., room temperature (rt) and 37° C., for 28 d. The absorbance of the SNAP-Si were measured by dissolving the material in THF (1 mg mL⁻¹) before and after the storage. (n=3 and error bars represent standard.

FIG. 4 shows the swelling ratio of silicone disks in different silicone oils. Silicone oils were dissolved in THF at 100 mg/mL. Disks were soaked in the solutions for designed time at −20° C., then taken out and dried in dark overnight before weighing. (n=3 and error bars represent standard deviations).

FIG. 5 shows the static contact angles of different disk samples. The contact angles were measured by Ossila Contact Angle Goniometer with 5 μL on disk surface. n=3 and error bars show standard deviations.

FIG. 6 shows the measurement of real-time NO release using a chemiluminescence nitric oxide analyzer (NOA). The NO flux levels were measured in PBS with 100 μM EDTA at 37° C. (n=3, and data represent mean±standard deviation).

FIG. 7 shows the antimicrobial effects of silicone disks treated with SNAP-Si and NAP-Si. (n≥3, and error bars represent standard deviation).

FIG. 8 shows a synthetic scheme for synthesizing RSNO—Si oils.

FIGS. 9A-9C show (A) FT-IR, (B) ¹H NMR and, (C) ¹³C NMR of RSNO1-Si and HS—Si oils. The FT-IR measurement was obtained at 2 cm⁻¹ resolution and 32 scans over the wavenumber range of 500-4000 cm⁻¹. NMR was obtained by a Varian/Agilent VNMRS 600 MHz, and ¹H and ¹³C NMR were reported in ppm relative to the internal solvent resonances of CDCl₃, with 64 and 216 scans, respectively.

FIG. 10 shows the final RSNO concentrations of RSNO—Si oils with different percentages of conversion. Data represents mean±standard deviation (n=3).

FIG. 11 shows the viscosity of RSNO—Si oils at rt (23° C.). Data represents mean±standard deviation (n=3).

FIGS. 12A-12B show the swelling ratio of disks in different oils at (A) rt (23° C.) and (B) in the freezer (−20° C.). Data represents mean±standard deviation (n=3).

FIG. 13 shows the contact angles of SR, RSNO0.1—Si—SR, RSNO0.5—Si—SR, RSNO1-Si—SR, and HS—Si—SR. Data represents mean±standard deviation (n=3).

FIGS. 14A-14F show NO-releasing profiles (A-C) and RSNO leaching (D-F) of the RSNO—Si—SR disks. (A) and (D) RSNO0.1—Si; (B) and (E) RSNO0.5—Si; (C) and (F) RSNO1-Si. NO release was measured by a chemiluminescence nitric oxide analyzer (NOA) under physiological conditions. The NO flux levels were measured in PBS with 100 μM EDTA at 37° C. Leaching of RSNO—Si from RSNO—Si—SR disks was measured by UV-vis at 340 nm. (n=3, and data represent mean±standard deviation).

FIG. 15 shows the antibacterial activity of RSNO—Si—SR calculated as a log of the colony forming units (CFU) cm⁻² of surface area against S. aureus bacteria. Data represents mean±standard error of mean (n≥3). * corresponds to p<0.05 calculated for SR vs. RSNO0.5—Si—SR and RSNO1-Si—SR, ** corresponds to p<0.01 calculated for SR vs. RSNO0.1—Si—SR.

FIGS. 16A-16C show the measurement of real-time NO release from SNAP—Si based polymer films using a chemiluminescence nitric oxide analyzer (NOA). (A) ssCB, (B) ssTF, and (C) ssTP. The NO flux levels were measured in PBS with 100 μM EDTA at 37° C. Data represent mean±standard deviation (n=3).

FIG. 17 shows the antibacterial activity of ssCB film calculated as a log of the colony forming units (CFU) cm⁻² of surface area against S. aureus bacteria. NAP-Si oil was added to CB to form NAP-CB as negative control. Data represents mean±standard error of mean.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a polysiloxane” include, but are not limited to, mixtures or combinations of two or more such polysiloxanes, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y”, where ‘x’ and ‘y’ are numerical values, includes “about ‘x’ to about ‘y”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated+10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance and instances where it does not.

As used herein, the term “biocompatible,” with respect to a substance or fluid described herein, indicates that the substance or fluid does not adversely affect the short-term viability or long-term proliferation of a target biological particle within a particular time range.

The terms “antimicrobial” and “antimicrobial characteristic” refer to the ability to kill and/or inhibit the growth of microorganisms. A substance having an antimicrobial characteristic may be harmful to microorganisms (e.g., bacteria, fungi, protozoans, algae, and the like). A substance having an antimicrobial characteristic can kill the microorganism and/or prevent or substantially prevent the growth or reproduction of the microorganism.

The term “antimicrobial effective amount” as used herein refers to that amount of the compound being administered which will kill microorganisms or inhibit growth and/or reproduction thereof to some extent (e.g. from about 5% to about 100%). In reference to the compositions or articles of the disclosure, an antimicrobial effective amount refers to that amount which has the effect of diminishment of the presence of existing microorganisms, stabilization (e.g., not increasing) of the number of microorganisms present, preventing the presence of additional microorganisms, delaying or slowing of the reproduction of microorganisms, and combinations thereof.

The terms “bacteria” or “bacterium” include, but are not limited to, gram positive and gram negative bacteria. Bacteria can include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anabaena affinis and other cyanobacteria (including the Anabaena, Anabaenopsis, Aphanizomenon, Camesiphon, Cylindrospermopsis, Gloeobacter Hapalosiphon, Lyngbya, Microcystis, Nodularia, Nostoc, Phormidium, Planktothrix, Pseudoanabaena, Schizothrix, Spirulina, Trichodesmium, and Umezakia genera) Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Eubacterium, Erwinia, Erysipelothrix, Escherichia, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Phytoplasma, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Spiroplasma, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholera, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof. The gram-positive bacteria may include, but is not limited to, gram positive Cocci (e.g., Streptococcus, Staphylococcus, and Enterococcus). The gram-negative bacteria may include, but is not limited to, gram negative rods (e.g., Bacteroidaceae, Enterobacteriaceae, Vibrionaceae, Pasteurellae and Pseudomonadaceae).

The term “antimicrobial effective amount” as used herein refers to that amount of the compound being administered/released that will kill microorganisms or inhibit growth and/or reproduction thereof to some extent (e.g. from about 5% to about 100%). In reference to the compositions or articles of the disclosure, an antimicrobial effective amount refers to that amount which has the effect of diminishment of the presence of existing microorganisms, stabilization (e.g., not increasing) of the number of microorganisms present, preventing the presence of additional microorganisms, delaying or slowing of the reproduction of microorganisms, and combinations thereof. Similarly, the term “antibacterial effective amount” refers to that amount of a compound being administered/released that will kill bacterial organisms or inhibit growth and/or reproduction thereof to some extent (e.g., from about 5% to about 100%). In reference to the compositions or articles of the disclosure, an antibacterial effective amount refers to that amount which has the effect of diminishment of the presence of existing bacteria, stabilization (e.g., not increasing) of the number of bacteria present, preventing the presence of additional bacteria, delaying or slowing of the reproduction of bacteria, and combinations thereof.

As used herein, the term “subject” includes humans, mammals (e.g., cats, dogs, horses, etc.), birds, and the like. Typical subjects to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications. In some embodiments, a system includes a sample and a host. The term “living host” refers to the entire host or organism and not just a part excised (e.g., a liver or other organ) from the living host.

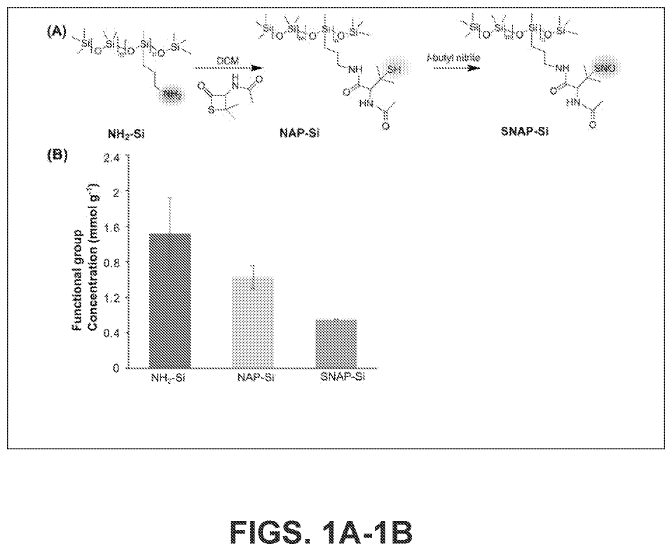

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O-units in the polyester, regardless of whether ethylene glycol was used to prepare the polyester. Referring to FIG. 1A, when the polysiloxane with pendant amino groups is reacted with the thiolactone NAP followed by nitrosylation, a residue of S-nitroso-N-acetyl-penicillamine is produced, where the residue covalently bonded to the polysiloxane. The residue of S-nitroso-N-acetyl-penicillamine is depicted below.

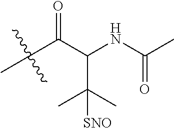

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In some embodiments, a straight chain or branched chain alkyl has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g. have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above having from one to ten carbons, or from one to six carbon atoms in its backbone structure. Likewise, “lower alkenyl” and “lower alkynyl” have similar chain lengths. In embodiments described in the present application, preferred alkyl groups are lower alkyls. In some embodiments, a substituent designated herein as alkyl is a lower alkyl.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

The term “alkylthio” refers to an alkyl group, as defined above, having a sulfur radical attached thereto. In some embodiments, the “alkylthio” moiety is represented by one of-S-alkyl, —S-alkenyl, and —S-alkynyl. Representative alkylthio groups include methylthio, and ethylthio. The term “alkylthio” also encompasses cycloalkyl groups, alkene and cycloalkene groups, and alkyne groups. “Arylthio” refers to aryl or heteroaryl groups. Alkylthio groups can be substituted as defined above for alkyl groups.

The terms “alkenyl” and “alkynyl”, refer to unsaturated aliphatic groups analogous in length and possible substitution to the alkyls described above, but that contain at least one double or triple bond respectively. The term “herteroalkenyl” is an alkenyl group substituted with one or more heteroatoms such as, for example, oxygen, nitrogen, or sulfur.

The terms “alkoxyl” or “alkoxy” as used herein refers to an alkyl group, as defined above, having an oxygen radical attached thereto. Representative alkoxyl groups include methoxy, ethoxy, propyloxy, and tert-butoxy. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl. The term “herteroalkoxy” is an alkoxy group substituted with one or more heteroatoms such as, for example, oxygen, nitrogen, or sulfur.

The terms “amine” and “amino” are art-recognized and refer to both unsubstituted and substituted amines, e.g., a moiety that can be represented by the general formula:

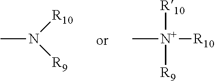

wherein R⁹, R¹⁰, and R¹⁰′ each independently represent a hydrogen, an alkyl, an alkenyl, —(CH₂)ₘ—R⁸ or R⁹ and R¹⁰ taken together with the N atom to which they are attached complete a heterocycle having from 4 to 8 atoms in the ring structure; R⁸ represents an aryl, a cycloalkyl, a cycloalkenyl, a heterocycle or a polycycle; and m is zero or an integer in the range of 1 to 8. In some embodiments, only one of R⁹ or R¹⁰ can be a carbonyl, e.g., R⁹, R¹⁰ and the nitrogen together do not form an imide. In still other embodiments, the term “amine” does not encompass amides, e.g., wherein one of R⁹ and R¹⁰ represents a carbonyl. In additional embodiments, R⁹ and R¹⁰ (and optionally R′10) each independently represent a hydrogen, an alkyl or cycloalkyl, an alkenyl or cycloalkenyl, or alkynyl. Thus, the term “alkylamine” as used herein means an amine group, as defined above, having a substituted (as described above for alkyl) or unsubstituted alkyl attached thereto, i.e., at least one of R₉ and R¹⁰ is an alkyl group.

The term “amido” is art-recognized as an amino-substituted carbonyl and includes a moiety that can be represented by the general formula:

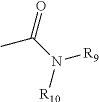

wherein R⁹ and R¹⁰ are as defined above.

“Aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro [2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “aralkyl”, as used herein, refers to an alkyl group substituted with an aryl group (e.g., an aromatic or heteroaromatic group).

The term “carbocycle”, as used herein, refers to an aromatic or non-aromatic ring in which each atom of the ring is carbon.

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, (C₁-C₁₀)alkyl, phenyl or benzyl, and optionally containing 1-3 double bonds and optionally substituted with one or more substituents. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro [2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents at one or more positions as defined above for alkyl and aryl, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, and —CN.

The term “carbonyl” is art-recognized and includes such moieties as can be represented by the general formula:

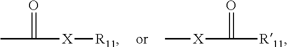

wherein X is a bond or represents an oxygen or a sulfur, and R¹¹ represents a hydrogen, an alkyl, a cycloalkyl, an alkenyl, an cycloalkenyl, or an alkynyl, R¹¹′ represents a hydrogen, an alkyl, a cycloalkyl, an alkenyl, an cycloalkenyl, or an alkynyl. Where X is an oxygen and R¹¹ or R¹¹′ is not hydrogen, the formula represents an “ester”. Where X is an oxygen and R¹¹ is as defined above, the moiety is referred to herein as a carboxyl group, and particularly when R¹¹ is a hydrogen, the formula represents a “carboxylic acid”. Where X is an oxygen and R¹¹′ is hydrogen, the formula represents a “formate”. In general, where the oxygen atom of the above formula is replaced by sulfur, the formula represents a “thiocarbonyl” group. Where X is a sulfur and R¹¹ or R¹¹′ is not hydrogen, the formula represents a “thioester.” Where X is a sulfur and R¹¹ is hydrogen, the formula represents a “thiocarboxylic acid.” Where X is a sulfur and R¹¹′ is hydrogen, the formula represents a “thioformate.” On the other hand, where X is a bond, and R¹¹ is not hydrogen, the above formula represents a “ketone” group. Where X is a bond, and R¹¹ is hydrogen, the above formula represents an “aldehyde” group.

The term “monoester” as used herein refers to an analogue of a dicarboxylic acid wherein one of the carboxylic acids is functionalized as an ester and the other carboxylic acid is a free carboxylic acid or salt of a carboxylic acid. Examples of monoesters include, but are not limited to, to monoesters of succinic acid, glutaric acid, adipic acid, suberic acid, sebacic acid, azelaic acid, oxalic and maleic acid.

The term “heteroatom” as used herein means an atom of any element other than carbon or hydrogen. Examples of heteroatoms include, but are not limited to boron, nitrogen, oxygen, phosphorus, sulfur and selenium. Other heteroatoms include silicon and arsenic.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms (for example, 1-14 carbon atoms), and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, peptide, and polypeptide groups.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. The heteroatoms such as nitrogen may have hydrogen substituents and/or any permissible substituents of organic compounds described herein which satisfy the valencies of the heteroatoms.

In various aspects, the substituent is selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

The term “copolymer” as used herein, generally refers to a single polymeric material that is comprised of two or more different monomers. The copolymer can be of any form, such as random, block, graft, etc. The copolymers can have any end-group, including capped or acid end groups.

The terms “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, delaying or slowing of disease progression, substantially preventing spread of disease, amelioration or palliation of the disease state, and remission (partial or total) whether detectable or undetectable.

The term “prevent” or “preventing” as used herein is defined as eliminating or reducing the likelihood of the occurrence of one or more symptoms of a disease or disorder (e.g., biofilm formation) when using the compositions as described herein when compared to a control where the composition is not used.

### Nitric Oxide Releasing Polysiloxanes and Methods of Making and Uses Thereof

Described herein are nitric oxide-releasing polysiloxanes. In one aspect, the nitric oxide-releasing polysiloxanes comprise a polysiloxane backbone with one or more nitric oxide releasing moieties pendant to the polysiloxane backbone. The nitric oxide-releasing polysiloxanes described herein provide highly sustained, long-term nitric oxide release that have numerous biological activities.

In one aspect, the nitric oxide releasing polysiloxane is produced the method comprising:
reacting a thiolactone with a polysiloxane comprising a polysiloxane backbone with one or more amino groups pendant to the polysiloxane backbone to produce a thiol-functionalized polysiloxane, and
nitrosating a thiol group on the thiol-functionalized polysiloxane to produce the nitric oxide releasing polysiloxane.

In one aspect, the polysiloxane backbone comprises a plurality of units having the structure II, where each R³ is an alkyl group.

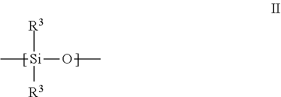

Here, the polysiloxane is a dialkyl polysiloxane. In one aspect, each R³ is the same alkyl group. For example, each R³ is methyl or ethyl.

In another aspect, the polysiloxane includes one more units having the structure III;

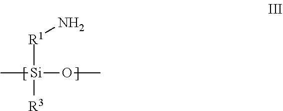

wherein R¹ is a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy; and R² is an alkyl group.

In this aspect, the amino groups in structure III are pendant to the polysiloxane backbone. In one aspect, R¹ in structure III is a substituted or unsubstituted C₁-C₁₂ alkyl group. In another aspect, R¹ in structure III is methylene, ethylene, propylene, or butylene. In one aspect, R² in structure III is a substituted or unsubstituted C₁-C₁₂ alkyl group. In another aspect, R² in structure III is methyl, ethyl, or propyl.

In one aspect, the polysiloxane comprising the polysiloxane backbone with one or more amino groups pendant to the polysiloxane backbone comprises a copolymer having a plurality of units of structure II and a plurality of units having the structure III. The relative amounts of structural units II and Ill present in the polysiloxane can be varied depending upon the number of pendant amino groups that are required or desired.

The polysiloxane comprising the polysiloxane backbone with one or more amino groups pendant to the polysiloxane backbone is reacted with a compound that possesses one or more sulfur groups that can be subsequently nitrosylated. In one aspect, the polysiloxane is reacted with a thiolactone. Not wishing to be bound by theory, the amino groups present in the polysiloxane react with the thiolactone, where the thiolactone ring-opens to produce a free thiol group or ion. In one aspect. In one aspect, the thiolactone has the structure:

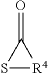

where R⁴ is a substituted or unsubstituted C₁-C₁₂ alkyl (e.g., methylene, ethylene, propylene, butylene).

In another aspect, the thiolactone has the structure:

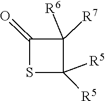

where each occurrence of R⁵ is independently hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, or a substituted or unsubstituted C₁-C₆ heteroalkoxy group;
R⁶ is hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, or a substituted or unsubstituted C₁-C₆ heteroalkoxy group; and
R⁷ is hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, a substituted or unsubstituted C₁-C₆ heteroalkoxy group, or an amide group of the formula —NHC(O)R⁸, wherein R⁸ is a substituted or unsubstituted C₁-C₆ alkyl group, substituted or unsubstituted C₁-C₆ heteroalkyl group.

In one aspect, the thiolactone is N-acetylcysteine thiolactone, N-acetyl-homocysteine thiolactone, homocysteine thiolactone, and butyryl-homocysteine thiolactone, or any combination thereof. In one aspect, the nitric oxide releasing material includes a plurality of-S—NO groups. For example, when the polysiloxane with pendant amino groups is reacted with a thiolactone as provided above, a plurality of thiol groups is produced. The thiol groups can subsequently be nitrosylated by reacting the free thiol groups with a nitrosylating agent. In one aspect, the nitrosylating agent is t-butyl nitrite, isopentyl nitrite, isobutyl nitrite, amyl nitrite, or cyclohexyl nitrite.

In one aspect, the nitric oxide releasing polysiloxane comprises one or more units having the structure I

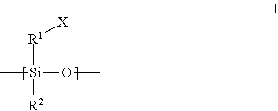

wherein R¹ is a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy;
R² is an alkyl group; and
X comprises a nitric oxide releasing moiety.

In one aspect, R¹ in structure I is a substituted or unsubstituted C₁-C₁₂ alkyl group. In another aspect, R¹ in structure I is methylene, ethylene, propylene, or butylene. In one aspect, R² in structure I is a substituted or unsubstituted C₁-C₁₂ alkyl group. In another aspect, R² in structure I is methyl, ethyl, or propyl.

In one aspect, the nitric oxide releasing moiety in structure I comprises a S-nitrosothiol compound. In another aspect, the nitric oxide-donating moiety in structure I is a residue of S-nitroso-N-acetyl-penicillamine, S-nitroso-N-acetyl, S-nitroso-N-acetyl cysteamine, S-nitrosoglutathione, methyl S-nitrosothioglycolate, and a derivative thereof.

In another aspect, nitric oxide releasing polysiloxane comprises a copolymer having a plurality of units of structure I and a plurality of units having the structure II. Non-limiting procedures for making the nitric oxide releasing polysiloxanes described herein are provided in the Examples, with FIG. 1A depicting a synthetic reaction scheme.

In another aspect, the nitric oxide releasing polysiloxane includes one or more units having the structure IV

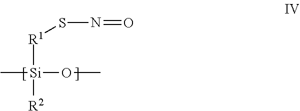

wherein R¹ is a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy; and
R² is an alkyl group.

In one aspect, R¹ in structure IV is a substituted or unsubstituted C₁-C₁₂ alkyl group. In another aspect, R¹ in structure IV is methylene, ethylene, propylene, or butylene. In another aspect, R² in structure IV is a substituted or unsubstituted C₁-C₁₂ alkyl group. In another aspect, R² in structure IV is methyl, ethyl, or propyl.

In one aspect, the nitric oxide releasing polysiloxane having the structure IV is produced the method comprising nitrosating a thiol group on a thiol-functionalized polysiloxane to produce the nitric oxide releasing polysiloxane. In one aspect, the thiol-functionalized polysiloxane comprises one more units having the structure V;

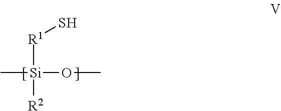

wherein R¹ is a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy; and R² is an alkyl group.

In one aspect, R¹ in structure V is a substituted or unsubstituted C₁-C₁₂ alkyl group. In another aspect, R¹ in structure V is methylene, ethylene, propylene, or butylene. In another aspect, R² in structure V is a substituted or unsubstituted C₁-C₁₂ alkyl group. In another aspect, R² in structure V is methyl, ethyl, or propyl.

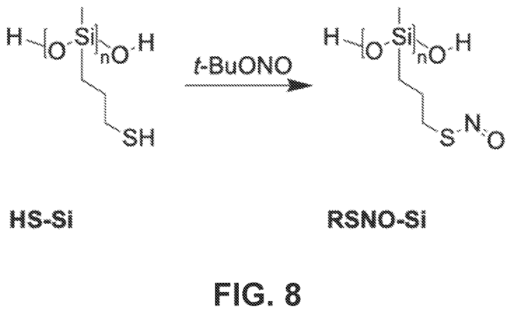

The thiol groups can subsequently be nitrosylated by reacting the free thiol groups with a nitrosylating agent. In one aspect, the nitrosylating agent is t-butyl nitrite, isopentyl nitrite, isobutyl nitrite, amyl nitrite, or cyclohexyl nitrite. FIG. 8 provides an exemplary reaction scheme for producing nitric oxide releasing polysiloxane having units of structure IV. Non-limiting procedures for making nitric oxide releasing polysiloxanes having these structural units are provided in the Examples.

The amount of the nitric oxide-donating moieties present in the nitric oxide releasing polysiloxane can vary. In one aspect, the nitric oxide-donating moieties are present in an amount from about 0.005 millimoles per gram of the polysiloxane to about 2.5 millimoles per gram of the polysiloxane, or about 0.005 millimoles per gram, 0.01 millimoles per gram, 0.05 millimoles per gram 0.1 millimoles per gram, 0.20 millimoles per gram, 0.25 millimoles per gram, 0.30 millimoles per gram, 0.35 millimoles per gram, 0.40 millimoles per gram, 0.45 millimoles per gram, 0.50 millimoles per gram, 0.55 millimoles per gram, 0.60 millimoles per gram, 0.65 millimoles per gram, 0.70 millimoles per gram, 0.75 millimoles per gram, 0.80 millimoles per gram, 0.85 millimoles per gram, 0.90 millimoles per gram, 0.95 millimoles per gram, 1.0 millimoles per gram, 1.25 millimoles per gram, 1.50 millimoles per gram, 1.75 millimoles per gram, 2.00 millimoles per gram, 2.25 millimoles per gram, or 2.50 millimoles per gram, where any value can be a lower and upper endpoint of range (e.g., 0.30 millimoles per gram to 0.70 millimoles per gram).

In certain aspects, the nitric oxide releasing polysiloxane is a viscous oil, which makes them useful as lubricants. In one aspect, the nitric oxide releasing polysiloxane has a kinematic viscosity of about 10 cSt to about 6,000 cSt, or about 10 cSt, 50 cSt, 100 cSt, 500 cSt, 1000 cSt, 1500 cSt, 2000 cSt, 2500 cSt, 3000 cSt, 3500 cSt, 4000 cSt, 4500 cSt, 5000 cSt, 5500 cSt, or 6000 cSt, where any value can be a lower and upper endpoint of range (e.g., 2,500 cSt to 5,000 cSt).

By varying the relative amount of the nitric oxide releasing moieties in the polysiloxane, the rate of release of the nitric oxide from the polysiloxane can be modified. In certain applications, it is desirable to have sustained release of nitric oxide from the polysiloxane under physiological conditions. In one aspect, nitric oxide is released from the polysiloxane up to two days at 37° C.

The nitric oxide releasing polysiloxanes described herein are useful in applications where it is desirable to reduce or prevent biofouling (e.g., bacterial adhesion, platelet formation, etc.) of implantable medical devices. Implantable medical devices are a leading cause of infection such as nosocomial infections. Implantable devices coated with or constructed of the compositions described herein can reduce or prevent biofouling in a subject when the device is introduced into the subject. In one aspect, the nitric oxide releasing polysiloxanes described herein can reduce or prevent bacterial growth on a surface of an implantable device. In another aspect, the nitric oxide releasing polysiloxanes described herein can reduce or prevent biofilm formation on a surface of an implantable device. In another aspect, the nitric oxide releasing polysiloxanes described herein can reduce or prevent fibrinogen formation on a surface of an implantable device.

In one aspect, the implantable device is a urinary catheter, artificial heart valve, a vascular catheter, a graft, or a stent. In other aspects, the device is intended to contact human blood or tissue. In one aspect, the device is a hemodialysis device or a component thereof.

The compositions described herein can be incorporated into devices in a number of different ways. In one aspect, the devices can be coated with the nitric oxide releasing polysiloxane as described herein. The coating of the device can be performed using techniques known in the art such as, for example, spraying or dipping the device with the nitric oxide releasing polysiloxane. The coating thickness can vary as well depending upon the device and application selected. In one aspect, the nitric oxide releasing polysiloxane coating has a thickness of from about 0.1 mm to about 5 mm, or about 0.1 mm, 0.5 mm, 1.0 mm, 1.5 mm, 2.0 mm, 2.5 mm, 3.0 mm, 3.5 mm, 4.0 mm, 4.5 mm, or 5.0 mm, where any value can be a lower and upper endpoint of range (e.g., 0.5 mm to 3.0 mm).

In another aspect, described herein are polymeric articles, wherein the nitric oxide releasing polysiloxanes described herein are homogeneously (i.e., evenly) dispersed throughout the polymer. In one aspect, the article is produced by (1) admixing the polymer and the nitric oxide releasing polysiloxane in a solvent to produce a first composition and (2) removing the solvent from the first composition to produce the article. The solvent used to blend the polymer and the nitric oxide releasing polysiloxane can vary depending upon the solubility of the components. In one aspect, the solvent is an organic solvent such as, for example, tetrahydrofuran, chloroform, methylene chloride, cyclohexanone, or any combination thereof. The amount of the nitric oxide releasing polysiloxane relative to the polymer can vary depending upon the amount of NO to be released.

After the nitric oxide releasing polysiloxane and the polymer have been admixed for a sufficient time, the solvent is removed to produce the article. In one aspect, the solution of the nitric oxide releasing polysiloxane and the polymer can be poured into a mold of any desired shape or dimensions prior to removal of the solvent can be removed using numerous techniques known in the art. Depending upon the selection of the solvent, heat and/or vacuum can be applied to solution to remove the solvent. In another embodiment, the solvent can be allowed to evaporate at room temperature.

In one aspect, the release rate of NO from the article can be tuned or modified based on the polymer's ability to uptake water. Not wishing to be bound by theory, NO release is regulated by the decomposition the nitric oxide releasing polysiloxane and generate thiyl radicals to trigger more reaction and NO release. When polymer has high water uptake, water diffuses into the matrices easily, and increases the free volume and mobility of the molecular chains, which enhances the chance of thiyl radicals to trigger further reactions and form crosslinking. In one aspect, the polymer has a water uptake of from about 0.5 wt % to about 5 wt %, or about 0.5 wt %, 1.0 wt %, 1.0 wt %, 1.5 wt %, 2.0 wt %, 2.5 wt %, 3.0 wt %, 3.5 wt %, 4.0 wt %, 4.5 wt %, or 5.0 wt %, where any value can be a lower and upper endpoint of a range (e.g., 1.0 wt % to 2.0 wt %).

In one aspect, the polymer is a medical grade polymer useful in medical applications and devices. In one aspect, the polymer is silicone rubber (SR), ELAST-EON™ E2As (a siloxane-base polyurethane elastomer commercially available from Aortech Biomaterials, Scoresby Victoria, Australia), CARBOSIL® (a thermoplastic silicone-polycarbonate-urethane commercially available from DSM Biomedical Inc., Berkeley, Calif.), and TECOFLEX™ SG80A and TECOPHILLIC™ SP-60D-60 (both polyurethanes commercially available from The Lubrizol Corporation, Wickliffe, Ohio)).

In other aspects, the nitric oxide releasing polysiloxanes described herein can be used to fabricate a device such as, for example, medical devices. Medical devices like cannulas, catheters, heart valves, endovascular stents, and joint prostheses, are widely used to treat diseases and improve the life quality of patients. However, the infection hurdles the applications of indwelling medical devices and implants. Infections are widely reported in indwelling medical devices and implants. Indwelling medical device infections are associated with various pathogens, such as Staphylococcus aureus, Staphylococcus epidermidis, and Escherichia coli ².

Infection not only disrupts the normal use and functionality of indwelling devices, but also brings health risks to patients and decreases their quality of life. One example is the infections during insulin pump use for Type 1 Diabetic (T1D) patients. T1D patients require lifetime exogenous insulin, so continuous subcutaneous insulin infusion (CSII) therapy is vital for them. In the CSII therapy, the insulin is infused into the patient's body through a subcutaneous cannula; however, the infection leads to insulin cannula failure. Therefore, cannulas and infusion sites are required to be replaced and rotated every 2-3 days to avoid the complication of the CSII such as infection and inflammation issues.

To prevent infection of indwelling medical devices like cannulas and catheters, the elimination of bacteria attachment on the surface is critical. The nitric oxide releasing polysiloxanes described herein and articles including the same (e.g., medical devices) address these issues by releasing NO at tunable rates and duration to reduce or prevent the growth of bacteria and likelihood of infection.

### ASPECTS

Aspect 1. A nitric oxide releasing polysiloxane comprising a polysiloxane backbone with one or more nitric oxide releasing moieties pendant to the polysiloxane backbone.

Aspect 2. The nitric oxide releasing polysiloxane of Aspect 1, wherein the polysiloxane backbone comprises a dialkyl polysiloxane.

Aspect 3. The nitric oxide releasing polysiloxane of Aspect 1 or 2, wherein the polysiloxane comprises one or more units having the structure I

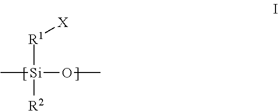

wherein R¹ is a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy;
R² is an alkyl group; and
X comprises a nitric oxide releasing moiety.

Aspect 4. The nitric oxide releasing polysiloxane of Aspect 3, wherein R¹ is a substituted or unsubstituted C₁-C₁₂ alkyl group.

Aspect 5. The nitric oxide releasing polysiloxane of Aspect 3, wherein R¹ is methylene, ethylene, propylene, or butylene.

Aspect 6. The nitric oxide releasing polysiloxane of any one of Aspects 3-5, wherein R² is a substituted or unsubstituted C₁-C₁₂ alkyl group.

Aspect 7. The nitric oxide releasing polysiloxane of any one of Aspects 3-5, wherein R² is methyl, ethyl, or propyl.

Aspect 8. The nitric oxide releasing polysiloxane of any one of Aspects 1-7, wherein the nitric oxide releasing moiety comprises a S-nitrosothiol compound.

Aspect 9. The nitric oxide releasing polysiloxane of any one of Aspects 1-7, wherein the nitric oxide-donating moiety is a residue of S-nitroso-N-acetyl-penicillamine, S-nitroso-N-acetyl cysteine, S-nitroso-N-acetyl cysteamine, S-nitrosoglutathione, methyl S-nitrosothioglycolate, and a derivative thereof.

Aspect 10. The nitric oxide releasing polysiloxane of any one of Aspects 1-9, wherein the polysiloxane comprises a copolymer having a plurality of units of structure I and a plurality of units having the structure II

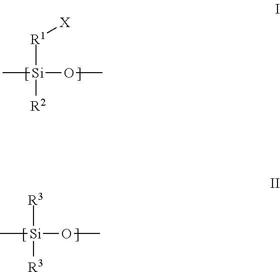

wherein R¹ is a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy;
R² is an alkyl group;
X comprises a nitric oxide releasing moiety; and
wherein R³ is an alkyl group.

Aspect 11. The nitric oxide releasing polysiloxane of Aspect 10, wherein R³ is a substituted or unsubstituted C₁-C₁₂ alkyl group.

Aspect 12. The nitric oxide releasing polysiloxane of Aspect 10, wherein R³ is methyl, ethyl, or propyl.

Aspect 13. The nitric oxide releasing polysiloxane of any of Aspects 1-12, wherein the nitric oxide releasing polysiloxane is produced the method comprising:
reacting a thiolactone with a polysiloxane comprising a polysiloxane backbone with one or more amino groups pendant to the polysiloxane backbone to produce a thiol-functionalized polysiloxane, and
nitrosating a thiol group on the thiol-functionalized polysiloxane to produce the nitric oxide releasing polysiloxane.

Aspect 14. The nitric oxide releasing polysiloxane of Aspect 13, wherein the polysiloxane backbone comprises a dialkyl polysiloxane.

Aspect 15. The nitric oxide releasing polysiloxane of Aspect 13 or 14, wherein the polysiloxane comprises one more units having the structure III;

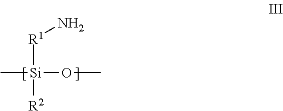

wherein R¹ is a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy; and R² is an alkyl group.

Aspect 16. The nitric oxide releasing polysiloxane of Aspect 15, wherein R¹ is a substituted or unsubstituted C₁-C₁₂ alkyl group.

Aspect 17. The nitric oxide releasing polysiloxane of Aspect 15, wherein R¹ is methylene, ethylene, propylene, or butylene.

Aspect 18. The nitric oxide releasing polysiloxane of any one of Aspects 15-17, wherein R² is a substituted or unsubstituted C₁-C₁₂ alkyl group.

Aspect 19. The nitric oxide releasing polysiloxane of any one of Aspects 15-17, wherein R² is methyl, ethyl, or propyl.

Aspect 20. The nitric oxide releasing polysiloxane of any one of Aspects 15-19, wherein the thiolactone has the structure

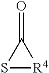

where R⁴ is a substituted or unsubstituted C₁-C₁₂ alkyl.

Aspect 21. The nitric oxide releasing polysiloxane of any one of Aspects 15-19, wherein the thiolactone has the structure

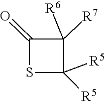

where each occurrence of R⁵ is independently hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, or a substituted or unsubstituted C₁-C₆ heteroalkoxy group;
R⁶ is hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, or a substituted or unsubstituted C₁-C₆ heteroalkoxy group; and
R⁷ is hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ herteroalkenyl group, a substituted or unsubstituted C₁-C₆ alkoxy group, a substituted or unsubstituted C₁-C₆ heteroalkoxy group, or an amide group of the formula —NHC(O)R⁸, wherein R⁸ is a substituted or unsubstituted C₁-C₆ alkyl group, substituted or unsubstituted C₁-C₆ heteroalkyl group.

Aspect 22. The nitric oxide releasing polysiloxane of any one of Aspects 15-19, wherein the thiolactone is selected from the group consisting of N-acetylcysteine thiolactone, N-acetyl-homocysteine thiolactone, homocysteine thiolactone, and butyryl-homocysteine thiolactone.

Aspect 23. The nitric oxide releasing polysiloxane of any one of Aspects 15-22, wherein the nitric oxide releasing moiety is a residue of S-nitroso-N-acetyl-penicillamine, S-nitroso-N-acetyl cysteine, S-nitroso-N-acetyl cysteamine, S-nitrosoglutathione, or methyl S-nitrosothioglycolate.

Aspect 24. The nitric oxide releasing polysiloxane of any one of Aspects 15-23, wherein the polysiloxane comprises a copolymer having a plurality of units having structures II and III.

Aspect 25. The nitric oxide releasing polysiloxane of Aspect 1 or 2, wherein the polysiloxane comprises one or more units having the structure IV

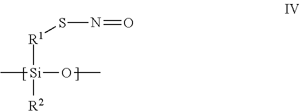

wherein R¹ is a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy; and R² is an alkyl group.

Aspect 26. The nitric oxide releasing polysiloxane of Aspect 25, wherein R¹ is a substituted or unsubstituted C₁-C₁₂ alkyl group.

Aspect 27. The nitric oxide releasing polysiloxane of Aspect 25, wherein R¹ is methylene, ethylene, propylene, or butylene.

Aspect 28. The nitric oxide releasing polysiloxane of any one of Aspects 25-27, wherein R² is a substituted or unsubstituted C₁-C₁₂ alkyl group.

Aspect 29. The nitric oxide releasing polysiloxane of any one of Aspects 25-27, wherein R² is methyl, ethyl, or propyl.

Aspect 30. The nitric oxide releasing polysiloxane of Aspect 1, wherein the nitric oxide releasing polysiloxane is produced the method comprising nitrosating a thiol group on a thiol-functionalized polysiloxane to produce the nitric oxide releasing polysiloxane.

Aspect 31. The nitric oxide releasing polysiloxane of Aspect 31, wherein the polysiloxane backbone comprises a dialkyl polysiloxane.

Aspect 32. The nitric oxide releasing polysiloxane of Aspect 30 or 31, wherein the thiol-functionalized polysiloxane comprises one more units having the structure V;

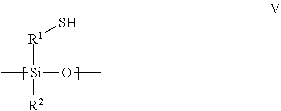

wherein R¹ is a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy; and R² is an alkyl group.

Aspect 33. The nitric oxide releasing polysiloxane of Aspect 32, wherein R¹ is a substituted or unsubstituted C₁-C₁₂ alkyl group.

Aspect 34. The nitric oxide releasing polysiloxane of Aspect 32, wherein R¹ is methylene, ethylene, propylene, or butylene.

Aspect 35. The nitric oxide releasing polysiloxane of any one of Aspects 32-34, wherein R² is a substituted or unsubstituted C₁-C₁₂ alkyl group.

Aspect 36. The nitric oxide releasing polysiloxane of any one of Aspects 32-34, wherein R² is methyl, ethyl, or propyl.

Aspect 37. The nitric oxide releasing polysiloxane of any of Aspects 1-36, wherein the nitric oxide-releasing moieties are present in an amount from about 0.005 millimoles per gram of the nitric oxide releasing polysiloxane to about 2.5 millimoles per gram of the nitric oxide releasing polysiloxane.

Aspect 38. The nitric oxide releasing polysiloxane of any of Aspects 1-37, wherein nitric oxide is released from the polysiloxane up to about two days at 37° C.

Aspect 39. An article comprising at least one surface, wherein the at least one surface is coated with the nitric oxide releasing polysiloxane of any one of Aspects 1-38.

Aspect 40. An article comprising one or more components fabricated with the nitric oxide releasing polysiloxane of any one of Aspects 1-38.

Aspect 41. The article of Aspect 40, wherein the one or more components comprises a polymer, wherein the nitric oxide releasing polysiloxane is homogeneously dispersed throughout the polymer.

Aspect 42. The article of Aspect 41, wherein the polymer comprises silicone rubber, a siloxane-base polyurethane elastomer, a polyurethane, or a thermoplastic silicone-polycarbonate-urethane.

Aspect 43. The article of Aspect 41, wherein the article is produced by (1) admixing the polymer and the nitric oxide releasing polysiloxane in a solvent to produce a first composition and (2) removing the solvent from the first composition to produce the article.

Aspect 44. The article of Aspect 43, wherein prior to step (2), pouring the first composition into a mold.

Aspect 45. The article of any of Aspects 39-44, wherein the article comprises a medical device.

Aspect 46. The article of Aspect 45, wherein the device is an implantable device.

Aspect 47. The article of Aspect 45, wherein the device is selected from the group consisting of: a vascular catheter, a urinary catheter, other catheters, a coronary stent, a wound dressing, and a vascular graft.

Aspect 48. A method of preventing bacterial growth on a surface of an article, the method comprising applying the nitric oxide releasing polysiloxane of any one of Aspects 1-38 to the surface.

Aspect 49. A method of preventing biofilm formation on a surface of an article, the method comprising applying the composition in any one of Aspects 1-38 to the surface.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

It should be emphasized that the above-described embodiments of the present disclosure are merely possible examples of implementations and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

### Example 1

### Materials and Methods

Materials. N-acetyl-D-penicillamine (NAP) was purchased from Sigma Aldrich (USA); plain S-nitroso-N-acetylpenicillamine (SNAP) was purchased from Pharmablock (China); t-butyl nitrite, tetrahydrofuran (THF), dichloromethane (DCM), pyridine, acetic anhydride, ninhydrin, acetic acid, ethanol, ethylenediaminetetraacetic acid (EDTA), and Tween 20 were purchased from Fisher Scientific (USA); phosphate buffer saline solution (10 mM PBS, pH 7.4) was prepared by the protocol from Cold Spring Harbor laboratory, and all ingredients (sodium phosphate dibasic, potassium phosphate monobasic, sodium chloride, potassium chloride) were purchased from Fisher Scientific; poly [dimethylsiloxane-co-(3-aminopropyl)methylsiloxane] (NH₂—Si, AMS-191) was purchased from Gelest (USA). Medical grade silicone sheet (87315K13) was purchased from McMaster-Carr (USA). All materials were used without purification unless mentioned specifically. Luria Bertani (LB) broth media and agar were purchased from Sigma Aldrich. Bacterial strains Escherichia coli (E. coli, ATCC 25922) and Staphylococcus aureus (S. aureus, ATCC 6538) were obtained from American Type Culture Collection (ATCC).

Synthesis and characterization of S-nitroso-N-acetylpenicillamine grafted silicone oil (SNAP-Si).

Synthesis of N-acetyl-D-penicillamine thiolactone (NAP-thiolactone). NAP-thiolactone was synthesized following a published method [46]. The initial amine concentration of aminate silicone oil (NH₂—Si) was determined by a modified ninhydrin assay [47]. Ninhydrin solution was prepared by dissolving ninhydrin (0.2 g) and acetic acid (0.5 mL) in ethanol (99.5 mL). The sample solution was prepared by dissolving sample (1 mg) in Tween 20 solution (1 mL, 1% w/v in H₂O); then, sample solution (0.5 mL), Tween 20 solution (0.5 mL) and ninhydrin solution (0.5 mL) were mixed and heated in boiling water for 10 min. After addition of ethanol (2.5 mL), the solution was checked by UV at 570 nm and the concentration was obtained by using a cysteine calibration curve.

Synthesis of SNAP-Si. SNAP-Si was synthesized by coupling NAP-thiolactone to poly [dimethylsiloxane-co-(3-aminopropyl)methylsiloxane] (NH₂—Si). NH₂—Si (5 g), dichloromethane (50 mL, DCM) and NAP-thiolactone (1.2 g) were placed in a 250 mL round bottom flask, and reacted overnight at room temperature (rt, 23° C.) to form NAP-Si. t-butyl nitrite was washed by equal volume of 20 mM cyclam vigorously to chelate trace metal, and the procedure was repeated for three times to obtain clean t-butyl nitrite [48]. Then, clean t-butyl nitrite (0.82 mL) and DCM (2 mL) was added to the NAP-Si and stirred at rt for 30 min to form green color solution, and condensed at 40° C. to remove excessive solvents. A green oil (SNAP-Si) was yielded, and it was stored in −20° C. freezer for further analysis.

Characterization of SNAP-Si by UV-vis, FT-IR and NMR. The successful synthesis of SNPA-Si was confirmed by UV-vis, Fourier transform infrared spectroscopy (FT-IR) and nuclear magnetic resonance (NMR). FT-IR spectra were taken by a PerkinElmer FT-IR Spectrum 3 spectrometer with KBr pellet. A drop of the sample was added to the KBr pellet and gently wiped by Kimwipe until a thin layer of oil was left on the surface. The FT-IR measurement was obtained at 2 cm⁻¹ resolution and 32 scans over the wavenumber range of 500-4000 cm⁻¹. NMR of SNAP-Si was obtained by a Varian/Agilent VNMRS 600 MHz with a 5 mm HCN cold probe and cooled carbon preamp. ¹H and ¹³C NMR were reported in ppm relative to the internal solvent resonances of CDCl₃, with 64 and 216 scans, respectively.

The SNAP concentration of SNAP-Si oil was quantified by a Cary 60 UV spectrometer using a SNAP calibration curve in THF. UV-vis spectra were taken within 300-500 nm wavelength at a medium scan speed. Commercial SNAP (Pharmablock, China) was dissolved in THF at the concentration of 0.1, 0.25, 0.5, 0.75 and 1 mM, then their absorbance at 340 nm was measured to make a calibration curve. To check the SNAP concentration in SNAP-Si oil, SNAP-Si was dissolved in THF (1 mg mL⁻¹ solution), and then its absorbance at 340 nm was measure and used to calculate the concentration of SNAP with the prior SNAP calibration curve.

SNAP-Si oil stability. The stability of SNAP-Si oil was monitored by checking quantity of the SNAP functionality at designed time points using UV-vis spectroscopy. SNAP-Si oil was placed in amber vials either in−20° C. freezer, rt, and 37° C. incubator to test the storage temperature stability for up to 4 weeks. At the designed timepoint, each sample was taken out and dissolved in THF at 1 mg mL⁻¹ concentration and measured for absorbance at 340 nm using UV-vis. The percentage of SNAP remaining on each day was quantified with respect to initial absorbance on the first day, and plotted overtime to show the storage stability under different temperature conditions.

Preparation of SNAP—Si—SR disks. A medical grade silicone sheet was punched into 0.7 cm diameter disks, and then soaked in the SNAP-Si oil or NAP-Si oil in THF (100 mg mL⁻¹) at −20° C. in dark. Samples were taken out and dried in fume hood for 12 h before weighing. The soaking process was monitored by swelling ratio, and the mass increase of samples was used to quantify the swelling. Weights of samples after swelling (wₜ) were compared with their original weights (wᵢ). The swelling ratio of each group was taken with the average of three samples, using the equation (1) below.

$$swelling ratio = \frac{{w}_{t} - {w}_{i}}{{w}_{i}} \times 100 \% \qquad ( 1 )$$

Water contact angles of disks. Static contact angles of samples were measured by an Ossila Contact Angle Goniometer (Ossila, UK). Sample disks were placed on the sample stage of contact angle goniometer, and 5 μL of deionized water were dropped on the surface. The static contact angles were measured from still frames using the sessile drop approximation, and the results were analyzed by Ossila Contact Angle Software. Three individual water droplets were placed at 3 different locations on the surface, and then averaged to get the average contact angles.

Leaching of the SNAP-Si oil from SNAP—Si—SR disks. In order to evaluate the risk of SNAP-Si leaching from the SR matrix, each SNAP—Si—SR disk was soaked in 1 mL of 10 mM PBS (pH 7.4) containing 100 μM EDTA at 37° C. in an incubator. To determine the amount of SNAP-Si oil leached out, the absorbance at 340 nm of the 1 mL of soaking solution was measured by UV-vis and the absorption was record. At the same time, the sample vial was replenished with 1 mL of fresh 10 mM PBS (pH 7.4, 100 μM EDTA), and incubated at 37° C. until the next reading.

NO release profiles of SNAP—Si—SR disks. NO released from the SNAP-Si treated disk samples was analyzed by a Zysense chemiluminescence Nitric Oxide Analyzer (NOA) 280i as previously reported [28], and the supply nitrogen flow rate and cell pressure were set up at 200 mL min⁻¹ and 8.8-9.5 psi, respectively. The sample vessel was partially submerged in a 37° C. water bath to mimic physiological temperature, and 3 mL of PBS buffer (10 mM, pH 7.4, containing 100 μM EDTA) was added to the reaction vessel. Each test started with a short period of baseline measurement, then SNAP—Si—SR disk was placed in the buffer within the sample vessel. NO released from the sample was purged by continuous N₂ flow and was detected in real-time by the chemiluminescence detector in 1 s interval until it reached stead-state. The NOA data was normalized with the surface area of samples to obtain the flux values with unit of mole cm⁻² min⁻¹. NO release was quantified at various time points during the experiment to track the release trends, and samples were incubated in PBS at 37° C. between each measurement.

Evaluating the antibacterial efficacy of SNAP—Si—SR disks. The antibacterial efficacy of SNAP—Si—SR disk samples was evaluated against two strains of bacteria Gram-positive (S. aureus) and Gram-negative (E. coli) in a 3 h antibacterial adhesion assay following previously reported protocol [49]. Briefly, an individual bacteria colony was inoculated in LB media and grown up to mid-log phase. Cells were extracted from mid-log phase and optical density of bacteria suspension was recorded using UV-vis spectroscopy at 600 nm wavelength. Then, cells were washed and resuspended in fresh media (final bacteria concentration=10⁷ CFU mL⁻¹) and exposed to UV-sterilized SR, NAP—Si—SR and SNAP—Si—SR disks for 3 h at 120 RPM, 37° C. After 3 h, samples were briefly rinsed to remove any loosely adhered bacteria on the films and resuspended in sterile PBS. To detach the adhered bacteria on the samples, bacteria exposed disks were homogenized and vortexed for 60 sec each. Bacteria suspension was then plated on LB agar plates using bacteria spiral plater (Eddy Jet 2W, IUL instruments). Plates were incubated at 37° C. overnight, and viable colonies on the LB agar plate were enumerated to determine the concentration of bacteria (CFU/mL) using colony counter (SphereFlash, IUL instruments). Antibacterial activity of SNAP—Si—SR disks compared to controls were determined using equation 1 and reported as CFU cm⁻² of polymer surface area.

$$\% bacterial reduction = \frac{[ ( Control ) - ( Test ) ] \times 100}{( Control )} \qquad ( 2 )$$

Statistical analysis. All the data were obtained with sample size ≥3, and data are reported as mean±standard deviation. A two-tailed Student's t-test with a hypothesis of unequal variance and α=0.05 were used to determine statistical significance.

### Results and Discussion

S-nitroso-N-acetyl-D-penicillamine grafted silicone oil (SNAP-Si) synthesis: The synthesis of SNAP-Si was achieved via two steps of synthesis: the first step is to graft NAP-thiolactone to the aminated silicone oil (NH₂—Si), followed by a second step of nitrosation of the thiol groups (FIG. 1A). This synthesis can be visualized directly due to the color changes. The starting NH₂—Si oil was transparent, but it turned green after the nitrosation due to green color of the tertiary S-nitrosothiol SNAP structure, indicating the successful grafting of SNAP moiety on the liquid silicone molecules. The initial amine concentration of NH₂—Si was determined by ninhydrin calibration curve, and the amine concentration of NH₂—Si was ˜1.5+0.41 mmol g⁻¹ (FIG. 1B). After coupling with NAP-thiolactone via a ring-opening reaction, NH₂—Si was converted to NAP-Si which contains the tertiary thiols. The residual amine concentration was ˜ 0.5+0.13 mmol g⁻¹ determined by ninhydrin test, which indicated ˜68% conversion to NAP and equaled ˜1.0+0.13 mmol g⁻¹ of thiols in NAP-Si. The second step of nitrosation resulted in green color silicone oil, and the SNAP concentration was quantified by UV with calibration curve. The final SNAP concentration was about 0.6 mmol g⁻¹ in SNAP-Si, which was about 54% conversion from NAP to SNAP.

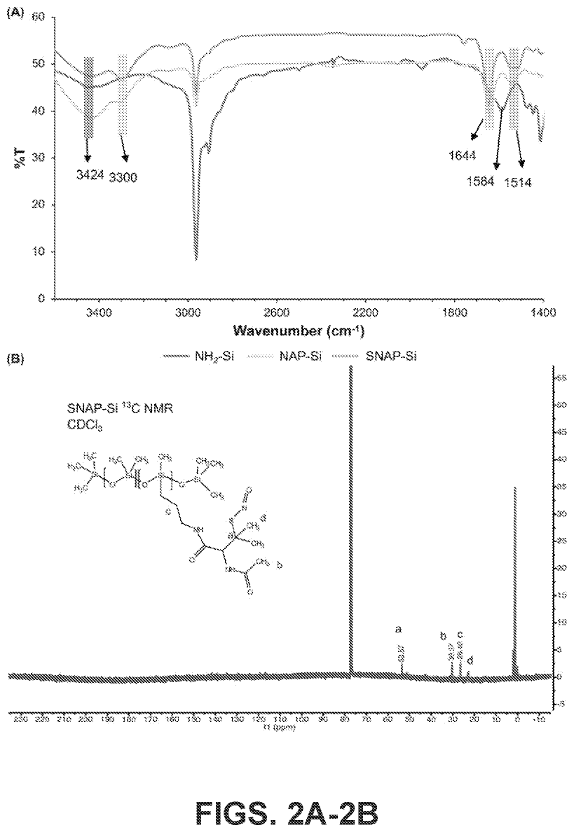

FT-IR and NMR were used to confirm the chemical structures of SNAP-Si. As shown in FIG. 2, NH₂—Si oil showed peak around 1584 cm⁻¹ which was assigned to primary amine N—H bending, and 3424 cm⁻¹ could be either H-bonded silanol or N—H stretching [50, 51]. For NAP-Si oil, the decrease of amine peak around 1584 cm⁻¹ and the appearance of new secondary amide peaks around 3300 cm⁻¹ suggested that NAP-thiolactone was coupled to the structure. For SNAP-Si oil, peak around 1644 cm⁻¹ and 1514 cm⁻¹ appeared to represent the secondary amide C═O stretching and the N—O stretching of the S-nitrosothiol, respectively [52, 53], demonstrating the SNAP moiety formation.

¹H and ¹³C NMR were performed to check product synthesis using an Agilent/Varian VNMR600 MHz instrument (FIG. 2B). Compared with NH₂—Si oil, 1H chemical shifts at 1.62, 1.85 and 2.04 ppm appeared in NAP-Si oil, indicating that NAP was tethered to the NH₂—Si oil via amide bonds. ¹³C chemical shifts were also observed at 22.74, 30.37, 51.29, 60.47, 169.53, and 170.32 ppm. Among these peaks, 169.53 and 170.32 ppm were assigned to carbonyls and confirmed the NAP on silicone. Even though the chemical shifts of 1H and ¹³C NMR for NAP-Si and SNAP-Si oils did not show dramatic changes before and after the nitrosation, a small change of ¹³C NMR was observed after the S-nitrosothiol formation. NAP-Si and SNAP-Si oils had ¹³C NMR chemical shifts at 51.29 and 53.57 ppm, respectively, and the 2.28 ppm shift to downfield could be induced by the nitroso group. It is noteworthy that in this study, SNAP moiety was grafted on silicone oil molecule, and the resulting SNAP-Si oil is a homogenous liquid that can be used as lubricant or to coat polymer directly and release NO gas.

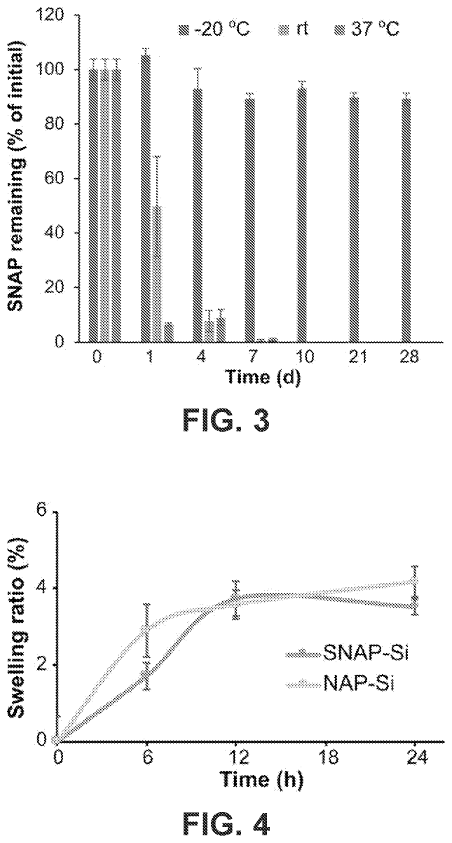

SNAP-Si oil stability. NO donors, like SNAP, hold great potential for biocompatibility and antibacterial applications. However, their rapid breakdown creates a major hindrance for therapeutic applications. SNAP can decompose in the presence of heat or light which restricts the life of NO release from material and adversely affect its clinical application [54, 55]. The shelf time of SNAP-Si oil is a critical issue; thus the temperature stability was studied by monitoring remaining SNAP concentration in SNAP-Si under different temperature (FIG. 3). Three different temperatures (rt, 37° C. and −20° C.) for up to 4 weeks were used to determine the best thermal conditions for potential storage and transport. The results from the study showed SNAP-Si oil samples were much more stable at −20° C. as compared to rt and 37° C. Data suggests that ˜ 89.21% of SNAP was present in the samples after 4 weeks of −20° C. storage relative to initial values obtained from samples on day 0. While the SNAP-Si oil seemed stable at −20° C., it was found less stable at rt and 37° C. with ca. 0.53 and 0.98% SNAP remaining after 7 days of storage, respectively. The instability of SNAP-Si oil at high temperature is due to the decomposition mechanism of SNAP, which is temperature dependent [56, 57]. As temperature increased, the thermal decomposition of S—N bond accelerates, generating more NO and sulfonyl radicals from the SNAP-Si, and made the SNAP-Si oil less stable [57]. Overall, the results obtained from the stability study reported here justify that SNAP-Si oil by itself permits long-term storage in colder temperature (−20° C.) with faster degradation of material at higher temperatures.

SNAP-Si oil infused silicone disks. SNAP—Si—SR and NAP—Si—SR disks were prepared by infusing SNAP-Si oil and NAP-Si oil on the surface, respectively. SNAP—Si—SR were light green due to the color of SNAP. To infuse same amount of SNAP-Si and NAP-Si oils to SR disks, the swelling ratio of SR disks were studied. Samples were taken out at designed time points during the soaking, then gently wiped by Kimwipe and weighed after completely dried. The increased weights represent the infused oil in the disks. As shown in FIG. 4, both samples showed the increasing trend during the infusion process. SNAP—Si—SR and NAP—Si—SR disks gained about 3.7% weight after 12 h of infusion.

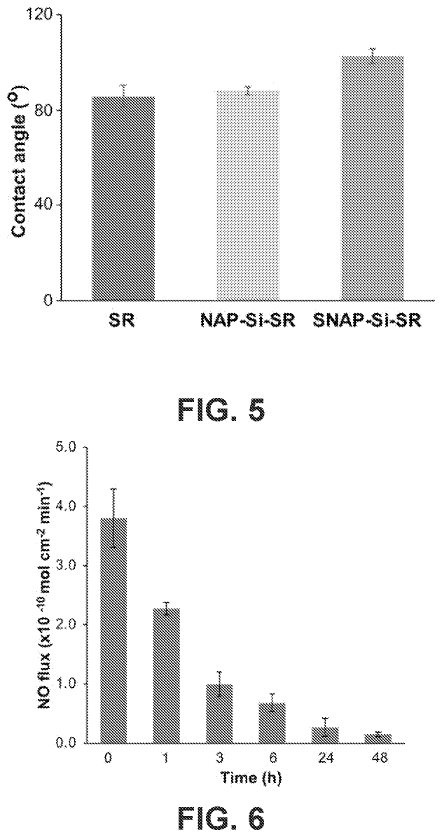

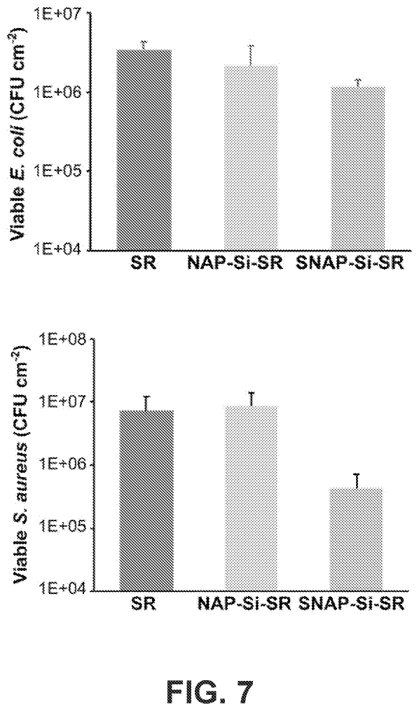

Static contact angles. The static contact angles of disks were determined by an Ossila Contact Angle Goniometer, and the static angles were shown in FIG. 5. Original commercial SR disks had static contact angle ˜ 86+4.5°, lower than 90° and demonstrated to be a slightly hydrophilic surface. It is interesting to find that while the contact angles of silicone materials range from hydrophilic (20-70°) to hydrophobic (95-122°) [34, 58-61], most of the silicone rubber (non-hydrogel) are hydrophobic, however, in our case the original SR disks had contact angles about 86°. Despite the experiment errors and other polymeric components which may affect the SR contact angles, a possible reason could be silicone oligomers on the SR surface. Usually, crosslinked SR rubbers have silicone oligomers on the surface, and these oligomers have been reported to cause the contact angle changes due to adaptive wetting behavior. The oligomers could absorb water form a thin oligomer-water lubricant layer, and at the same time the absorbed water could pull more oligomers from the matrix to enhance lubricant layer. Due to the existence of oligomer-water lubricant layer, the contact angle would decrease [62]. Therefore, our lower surface contact angle may relate to more oligomers on the SR surface than SRs in other papers. The NAP—Si—SR had static contact angle about 88+1.6°, indicating that surface infusion of NAP-Si oil had almost no changes on SR disk surface hydrophilicity. However, the static contact angle of SNAP—Si—SR was 103+3.1°, showing that SNAP-Si oil increased the hydrophobicity of SR surface. These results suggested that the SNAP—Si—SR surface became more hydrophobic than NAP—Si—SR, and SNAP—Si—SR disks had similar hydrophobicity like SNAP impregnated silicone materials (104-110)°. The different contact angles of NAP—Si—SR and SNAP—Si—SR may attribute to their chemical structures. The thiol groups in NAP-Si oil are hydrophilic, but many of thiols are converted to the S-nitrosothiol groups after the nitrosation to form SNAP-Si oil. So compared to NAP-Si, SNAP-Si has a smaller number of hydrophilic thiols, which may lead to the more hydrophobic surface of SNAP—Si—SR.

NO Release and SNAP Leaching of SNAP—Si—SR disks. The NO release profiles of the SNAP—Si—SR disks were measured by a Zysense chemiluminescence NOA 280i at 37° C. while incubated in 3 mL of PBS buffer (10 mM, pH 7.4, with 100 μM EDTA) [32]. The NO release profiles are shown in FIG. 6. The SNAP—Si—SR was able to release NO at physiologically relevant levels (3.8-0.7×10⁻¹⁰ mol min⁻¹ cm⁻¹) for >6 h, and then continued the low flux level (0.7-0.2×10⁻¹⁰ mol min⁻¹ cm⁻¹) up to 2 d until the NO payload was depleted. This high NO flux during the initial period and the gradually decreased NO release trend is commonly seen in other NO-releasing materials [27, 31]. In addition, the leaching of NO donors is always a safety concern for NO-releasing materials. Thus, the leaching of SNAP-Si oil from SNAP—Si—SR was analyzed using UV-vis spectroscopy (Figure S4). No SNAP-Si oil was detected in PBS buffer, suggesting no SNAP leaching during the NO release period and no inferences of leachates during antibacterial studies.

Bacterial assays of SNAP—Si—SR disks. The antibacterial properties of the SNAP—Si—SR samples and controls were analyzed in 3 h bacterial adhesion assay against S. aureus and E. coli (n≥3). The bacterial cells adhered on the surface of films were enumerated and normalized to the surface area of the disks (CFU cm⁻²). The NAP—Si—SR control disks showed ˜ 37.05% reduction of viable E. coli, while no reduction of S. aureus. SNAP—Si—SR disks exhibited 94.06 and 66.05% reduction in viable S. aureus and E. coli, respectively, compared to SR control due to the action of NO. SNAP—Si—SR disks killed both S. aureus and E. coli bacteria, and the results corroborate with other NO releasing surfaces previously reported that exhibit similar antibacterial activities [31, 63-65]. However, compare to SNAP—Si—SR disks, NAP—Si—SR disks showed weak reduction of E. coli adhesion and failed to reduce S. aureus adhesion; and the only structural difference was the SNO group which actively releases NO. Therefore, antibacterial effects of SNAP—Si—SR were majorly attributed to the active NO release from the surface. NO is also known to eradicate bacteria by non-specific processes which include formation of N₂O₃ and NO₃⁻¹ upon interaction with oxygen, and these oxidative molecules trigger cleavage of bacterial DNA and membranal damage [66].

Previous reports on SNAP-based biomaterials have demonstrated the antimicrobial effects against various pathogenic bacteria and fungus strains in medical device related infections such as P. aeruginosa, S. epidermidis, C. albicans etc [45, 67-69]. Nevertheless, these studies involved either blending or impregnating the materials with NO donor SNAP, which are often challenged because it delocalizes the NO release from the surfaces. While published studies used pure SNAP directly had leaching issues, grafting SNAP moiety to silicone oil avoided the leaching as SNAP-Si oil is not miscible with water, and no SNAP pattern peak was observed in UV-vis spectra at 340 nm. Besides, compared to traditional antibiotic and metal ion releasing surfaces, NO releasing surfaces not only inhibit the bacterial adhesion and kill bacteria without causing resistance and toxicity, but also serve as hemocompatible surfaces [25, 70-72]. Overall, the antibacterial results from this study and the previous success of NO-releasing materials as well as the non-leaching behavior of SNAP-Si in solutions suggests that SNAP-Si oil can be a promising approach to solving bacterial infections. For example, by soaking the insulin cannulas in SNAP-Si oil to infuse a thin layer in the cannula surface, the bacterial infection of insulin cannulas could be reduced due to the antibacterial effects of NO released from SNAP-Si. Similarly, SNAP-Si may be used to treat other medical device surface to eliminate the bacterial infection.

### Conclusions

In this study, SNAP-Si oil was successfully synthesized by grafting NO donor to silicone oil and confirmed by FT-IR and NMR. SNAP-Si oil was stable in−20° C. freezer storage, and more than 89% of SNAP was preserved after 4 weeks of storage. When SNAP-Si oil was infused into SR disk, the SNAP—Si—SR released 3.8×10⁻¹⁰ mol min⁻¹ cm⁻¹ of NO in the 1 h, and the NO release continued for up to 2 days without any leaching. The antibacterial effect of SNAP-Si was tested by 3 h bacterial adhesion assay against S. aureus and E. coli, and SNAP-Si lead to 94.06 and 66.05% reduction in viable S. aureus and E. coli, respectively. Results of this study suggested that SNAP-Si is a new NO-releasing silicone-based material which demonstrated antibacterial effects on both Gram-positive and Gram-negative bacteria. The ease of synthesis and excellent antibacterial effects against both Gram-positive and Gram-negative bacteria without resistance and serious leaching concerns, SNAP-Si oil could be potentially used for the development of antibacterial coating or lubricant for medical devices (e.g. cannulas, catheters, tubing, and etc.) in near future.

### REFERENCES FOR EXAMPLE 1

[1] C. R. Arciola, D. Campoccia, L. Montanaro, Implant infections: Adhesion, biofilm formation and immune evasion, Nat. Rev. Micro. 16 (2018) 397-409.

[2] A. Fusco, L. Coretti, V. Savio, E. Buommino, F. Lembo, G. Donnarumma, Biofilm formation and immunomodulatory activity of Proteus mirabilis clinically isolated strains, Int. J. Mol. Sci. 18 (2017) 414.

[3] F. Rahimi, M. Katouli, S. Karimi, Biofilm production among methicillin resistant Staphylococcus aureus strains isolated from catheterized patients with urinary tract infection, Microb. Pathog. 98 (2016) 69-76.

[4] M. Jamal, W. Ahmad, S. Andleeb, F. Jalil, M. Imran, M. A. Nawaz, T. Hussain, M. Ali, M. Rafiq, M. A. Kamil, Bacterial biofilm and associated infections, J. Chin. Med. Assoc. 81 (2018) 7-11.

[5] J. W. Costerton, Bacterial biofilms: A common cause of persistent infections, Science 284 (1999) 1318-1322.

[6] The top 10 causes of death, 2020. https://www.who.int/news-room/fact-sheets/detail/the-top-10-causes-of-death. (Accessed May 1 2021).

[7] L. Zhang, S. Cao, N. Marsh, G. Ray-Barruel, J. Flynn, E. Larsen, C. M. Rickard, Infection risks associated with peripheral vascular catheters, J. Infect. Prev. 17 (2016) 207-213.

[8] C. Beloin, N. Fernandez-Hidalgo, D. Lebeaux, Understanding biofilm formation in intravascular device-related infections, Intensive Care Med. 43 (2017) 443-446.

[9] O. Diaconu, I. Siriopol, L. I. Polosanu, I. Grigoras, Endotracheal tube biofilm and its impact on the pathogenesis of ventilator-associated pneumonia, J. Crit. Care Med. 4 (2018) 50-55.

C.-Y. Tan, N.-C. Chiu, K.-S. Lee, H. Chi, F.-Y. Huang, D. T.—N. Huang, L. Chang, Y.-H. Kung, C.-Y. Huang, Respiratory tract infections in children with tracheostomy, J. Microbiol. Immunol. Infect. 53 (2020) 315-320.

N. A. Juma, S. J. Forsythe, Microbial biofilm development on neonatal enteral feeding tubes, Springer International Publishing 2015, pp. 113-121.

P. Barbadoro, C. Marmorale, C. Recanatini, G. Mazzarini, I. Pellegrini, M. M. D′Errico, E. Prospero, May the drain be a way in for microbes in surgical infections?, Am. J. Infect. Control 44 (2016) 283-288.

B. Pittet, D. Montandon, D. Pittet, Infection in breast implants, Lancet Infect. Dis. 5 (2005) 94-106.

Type 1 diabetes facts, 2021. https://www.jdrf.org/t1d-resources/about/facts/. (Accessed Jun. 21 2021).

T. S. Guinn, G. J. Bailey, R. S. Mecklenburg, Factors related to discontinuation of continuous subcutaneous insulin-infusion therapy, Diabetes Care 11 (1988) 46-51.

R. S. Mecklenburg, E. A. Benson, J. W. Benson, Jr., P. N. Fredlund, T. Guinn, R. J. Metz, R. L. Nielsen, C. A. Sanner, Acute complications associated with insulin infusion pump therapy. Report of experience with 161 patients, JAMA 252 (1984) 3265-3269.

A. Guenego, G. Bouzille, S. Breitel, A. Esvant, J.-Y. Poirier, F. Bonnet, I. Guilhem, Insulin pump failures: Has there been an improvement? Update of a prospective observational study, Diabetes Technol. Ther. 18 (2016) 820-824.

R. J. Fair, Y. Tor, Antibiotics and bacterial resistance in the 21st century, Perspect Medicin. Chem. 6 (2014) 25-64.

P. Vallance, Nitric oxide: Therapeutic opportunities, Fundam. Clin. Pharmacol. 17 (2003) 1-10.

Y. Qian, J. B. Matson, Gasotransmitter delivery via self-assembling peptides: Treating diseases with natural signaling gases, Adv. Drug Deliv. Rev. 110-111 (2016) 137-156.

M. R. Garren, M. Ashcraft, Y. Qian, M. Douglass, E. J. Brisbois, H. Handa, Nitric oxide and viral infection: Recent developments in antiviral therapies and platforms, Appl. Mater. Today 22 (2021) 100887.

A. W. Carpenter, M. H. Schoenfisch, Nitric oxide release: Part II. Therapeutic applications, Chem. Soc. Rev. 41 (2012) 3742-3752.

A. Ghaffari, C. C. Miller, B. McMullin, A. Ghahary, Potential application of gaseous nitric oxide as a topical antimicrobial agent, Nitric Oxide 14 (2006) 21-29.

D. O. Schairer, J. S. Chouake, J. D. Nosanchuk, A. J. Friedman, The potential of nitric oxide releasing therapies as antimicrobial agents, Virulence 3 (2012) 271-279.

B. J. Privett, A. D. Broadnax, S. J. Bauman, D. A. Riccio, M. H. Schoenfisch, Examination of bacterial resistance to exogenous nitric oxide, Nitric Oxide 26 (2012) 169-173.

M. J. R. Ahonen, J. M. Dorrier, M. H. Schoenfisch, Antibiofilm efficacy of nitric oxide-releasing alginates against cystic fibrosis bacterial pathogens, ACS Infect. Dis. 5 (2019) 1327-1335.

E. J. Brisbois, J. Bayliss, J. Wu, T. C. Major, C. Xi, S. C. Wang, R. H. Bartlett, H. Handa, M. E. Meyerhoff, Optimized polymeric film-based nitric oxide delivery inhibits bacterial growth in a mouse burn wound model, Acta Biomater. 10 (2014) 4136-4142.

C. G. Feit, M. K. Chug, E. J. Brisbois, Development of S-nitroso-N-acetylpenicillamine impregnated medical grade polyvinyl chloride for antimicrobial medical device interfaces, ACS Appl. Bio Mater. 2 (2019) 4335-4345.

G. Lautner, M. E. Meyerhoff, S. P. Schwendeman, Biodegradable poly(lactic-co-glycolic acid) microspheres loaded with S-nitroso-N-acetyl-d-penicillamine for controlled nitric oxide delivery, J. Control. Release 225 (2016) 133-139.

A. Mondal, M. Douglass, S. P. Hopkins, P. Singha, M. Tran, H. Handa, E. J. Brisbois, Multifunctional S-nitroso-N-acetylpenicillamine-incorporated medical-grade polymer with selenium interface for biomedical applications, ACS Appl. Mater. Interfaces 11 (2019) 34652-34662.

E. J. Brisbois, H. Handa, T. C. Major, R. H. Bartlett, M. E. Meyerhoff, Long-term nitric oxide release and elevated temperature stability with S-nitroso-N-acetylpenicillamine (SNAP)-doped Elast-eon E2As polymer, Biomaterials 34 (2013) 6957-6966.

M. K. Chug, C. Feit, E. J. Brisbois, Increasing the lifetime of insulin cannula with antifouling and nitric oxide releasing properties, ACS Appl. Bio Mater. 2 (2019) 5965-5975.

J. A. González Calderón, D. Contreras López, E. Pérez, J. Vallejo Montesinos, Polysiloxanes as polymer matrices in biomedical engineering: Their interesting properties as the reason for the use in medical sciences, Polym. Bull. 77 (2020) 2749-2817.

A. Mata, A. J. Fleischman, S. Roy, Characterization of polydimethylsiloxane (PDMS) properties for biomedical micro/nanosystems, Biomed. Microdevices 7 (2005) 281-293.

H. S. El-Zaim, J. P. Heggers, Silicones for pharmaceutical and biomedical applications, Polymeric Biomaterials, Revised and Expanded, 2nd ed., Taylor & Francis Group (2001) pp 79-88.

M. N. Dimitrova, J. S. Bee, L. Lu, J. E. Fernandez, Development of prefilled syringe combination products for biologics, Springer International Publishing 2018, pp. 203-224.

W. Yuan, Z. Hu, M. Liao, Y. Cai, M. Lele, Z. Liu, Y. Chen, Y. Liu, N. Lu, A novel preparation method for silicone oil nanoemulsions and its application for coating hair with silicone, Int. J. Mol. Nanomedicine (2012) 5719.

T. Aziz, H. Fan, F. U. Khan, M. Haroon, L. Cheng, Modified silicone oil types, mechanical properties and applications, Polym. Bull. 76 (2019) 2129-2145.

G. E. Gierke, M. Nielsen, M. C. Frost, S-nitroso-N-acetyl-D-penicillamine covalently linked to polydimethylsiloxane (SNAP-PDMS) for use as a controlled photoinitiated nitric oxide release polymer, Sci. Technol. Adv. Mater. 12 (2011) 055007.

E. J. Brisbois, T. C. Major, M. J. Goudie, R. H. Bartlett, M. E. Meyerhoff, H. Handa, Improved hemocompatibility of silicone rubber extracorporeal tubing via solvent swelling-impregnation of S-nitroso-N-acetylpenicillamine (SNAP) and evaluation in rabbit thrombogenicity model, Acta Biomater. 37 (2016) 111-119.

G. Lautner, B. Stringer, E. J. Brisbois, M. E. Meyerhoff, S. P. Schwendeman, Controlled light-induced gas phase nitric oxide release from S-nitrosothiol-doped silicone rubber films, Nitric Oxide 86 (2019) 31-37.

J. Li, E. Ueda, D. Paulssen, P. A. Levkin, Slippery lubricant-infused surfaces: Properties and emerging applications, Adv. Funct. Mater. 29 (2019) 1802317.

N. MacCallum, C. Howell, P. Kim, D. Sun, R. Friedlander, J. Ranisau, O. Ahanotu, J. J. Lin, A. Vena, B. Hatton, T.-S. Wong, J. Aizenberg, Liquid-infused silicone as a biofouling-free medical material, ACS Biomater. Sci. Eng. 1 (2014) 43-51.

C. Wei, G. Zhang, Q. Zhang, X. Zhan, F. Chen, Silicone oil-infused slippery surfaces based on sol-gel process-induced nanocomposite coatings: A facile approach to highly stable bioinspired surface for biofouling resistance, ACS Appl. Mater. Interfaces 8 (2016) 34810-34819.

M. J. Goudie, J. Pant, H. Handa, Liquid-infused nitric oxide-releasing (LINOrel) silicone for decreased fouling, thrombosis, and infection of medical devices, Sci. Rep. 7 (2017) 13623.

S. P. Hopkins, J. Pant, M. J. Goudie, C. Schmiedt, H. Handa, Achieving long-term biocompatible silicone via covalently immobilized S-nitroso-N-acetylpenicillamine (SNAP) that exhibits 4 months of sustained nitric oxide release, ACS Appl. Mater. Interfaces 10 (2018) 27316-27325.

S. Ghalei, S. Hopkins, M. Douglass, M. Garren, A. Mondal, H. Handa, Nitric oxide releasing halloysite nanotubes for biomedical applications, J. Colloid Interface Sci. 590 (2021) 277-289.

M. J. Goudie, P. Singha, S. P. Hopkins, E. J. Brisbois, H. Handa, Active release of an antimicrobial and antiplatelet agent from a nonfouling surface modification, ACS Appl. Mater. Interfaces 11 (2019) 4523-4530.

M. K. Chug, E. Bachtiar, N. Narwold, K. Gall, E. J. Brisbois, Tailoring nitric oxide release with additive manufacturing to create antimicrobial surfaces, Biomater. Sci. (2021).

D. Chen, X. Hu, H. Zhang, X. Yin, Y. Zhou, Preparation and properties of novel polydimethylsiloxane composites using polyvinylsilsesquioxanes as reinforcing agent, Polym. Degrad. Stabil. 111 (2015) 124-130.

S. Ghalei, J. Li, M. Douglass, M. Garren, H. Handa, Synergistic approach to develop antibacterial electrospun scaffolds using honey and S-nitroso-N-acetyl penicillamine, ACS Biomater. Sci. Eng. 7 (2021) 517-526.

S. P. Hopkins, M. C. Frost, S-nitroso-N-acetyl-D-penicillamine modified hyperbranched polyamidoamine for high-capacity nitric oxide storage and release, Bioengineering 7 (2020).

C. Zhang, T. D. Biggs, N. O. Devarie-Baez, S. Shuang, C. Dong, M. Xian, S-nitrosothiols: Chemistry and reactions, Chem. Commun. 53 (2017) 11266-11277.

P. G. Wang, M. Xian, X. Tang, X. Wu, Z. Wen, T. Cai, A. J. Janczuk, Nitric oxide donors: Chemical activities and biological applications, Chem. Rev. 102 (2002) 1091-1134.

N. Hogg, Biological chemistry and clinical potential of S-nitrosothiols, Free Radical Biol. Med. 28 (2000) 1478-1486.

D. L. H. Williams, The chemistry of S-nitrosothiols, Acc. Chem. Res. 32 (1999) 869-876.

L. Grossi, P. C. Montevecchi, A kinetic study of S-nitrosothiol decomposition, Chem. Eur. J. 8 (2002) 380-387.

M. L. Read, P. B. Morgan, J. M. Kelly, C. Maldonado-Codina, Dynamic contact angle analysis of silicone hydrogel contact lenses, J. Biomater. Appl. 26 (2011) 85-99.

S. Vudayagiri, M. D. Junker, A. L. Skov, Factors affecting the surface and release properties of thin polydimethylsiloxane films, Polym. J. 45 (2013) 871-878.

Y. Wo, L.-C. Xu, Z. Li, A. J. Matzger, M. E. Meyerhoff, C. A. Siedlecki, Antimicrobial nitric oxide releasing surfaces based on S-nitroso-N-acetylpenicillamine impregnated polymers combined with submicron-textured surface topography, Biomater. Sci. 5 (2017) 1265-1278.

M. J. Goudie, E. J. Brisbois, J. Pant, A. Thompson, J. A. Potkay, H. Handa, Characterization of an S-nitroso-N-acetylpenicillamine-based nitric oxide releasing polymer from a translational perspective, Int. J. Polym. Mater. 65 (2016) 769-778.

W. S. Y. Wong, L. Hauer, A. Naga, A. Kaltbeitzel, P. Baumli, R. Berger, M. D′Acunzi, D. Vollmer, H.-J. Butt, Adaptive wetting of polydimethylsiloxane, Langmuir 36 (2020) 7236-7245.

J. Pant, M. J. Goudie, S. P. Hopkins, E. J. Brisbois, H. Handa, Tunable nitric oxide release from S-nitroso-N-acetylpenicillamine via catalytic copper nanoparticles for biomedical applications, ACS Appl. Mater. Interfaces 9 (2017) 15254-15264.

E. J. Brisbois, R. P. Davis, A. M. Jones, T. C. Major, R. H. Bartlett, M. E. Meyerhoff, H. Handa, Reduction in thrombosis and bacterial adhesion with 7 day implantation of S-nitroso-N-acetylpenicillamine (SNAP)-doped Elast-eon E2As catheters in sheep, J. Mater. Chem. B 3 (2015) 1639-1645.

C. Feit, M. K. Chug, E. J. Brisbois, Development of S-nitroso-N-acetylpenicillamine (snap) impregnated medical grade polyvinyl chloride for antimicrobial medical device interfaces, ACS Applied Bio Materials (2019).

F. C. Fang, Antimicrobial reactive oxygen and nitrogen species: Concepts and controversies, Nat. Rev. Microbiol. 2 (2004) 820.

H. Ren, J. Wu, A. Colletta, M. E. Meyerhoff, C. Xi, Efficient eradication of mature Pseudomonas aeruginosa biofilm via controlled delivery of nitric oxide combined with antimicrobial peptide and antibiotics, Front Microbiol. 7 (2016).

Y. Wo, E. J. Brisbois, J. Wu, Z. Li, T. C. Major, A. Mohammed, X. Wang, A. Colletta, J. L. Bull, A. J. Matzger, C. Xi, R. H. Bartlett, M. E. Meyerhoff, Reduction of thrombosis and bacterial infection via controlled nitric oxide (NO) release from S-nitroso-N-acetylpenicillamine (SNAP) impregnated carbosil intravascular catheters, ACS Biomater. Sci. Eng. 3 (2017) 349-359.

C. Macherla, D. A. Sanchez, M. Ahmadi, E. M. Vellozzi, A. J. Friedman, J. D. Nosanchuk, L. R. Martinez, Nitric oxide releasing nanoparticles for treatment of Candida albicans burn infections, Front Microbiol. 3 (2012) 193.

S. M. Kim Y S, Park J D, et al., Subchronic oral toxicity of silver nanoparticles, Part. Fibre Toxicol. (2010) 20.

T. Zhang, L. Wang, Q. Chen, C. Chen, Cytotoxic potential of silver nanoparticles, Yonsei Med. J. 55 (2014) 283-291.

A. Mondal, M. Douglass, S. P. Hopkins, P. Singha, M. Tran, H. Handa, E. J. Brisbois, Multifunctional S-nitroso-N-acetylpenicillamine incorporated medical grade polymer with selenium interface for biomedical applications, ACS Appl. Mater. Interfaces (2019).

### Example 2

Materials. t-butyl nitrite, tetrahydrofuran (THF), cyclam, ethylenediaminetetraacetic acid (EDTA) were purchased from Fisher Scientific; phosphate buffer saline (0.01 M, pH 7.4, containing 0.138 M NaCl, 2.7 mM KCl) was purchased from Sigma Aldrich; (mercaptopropyl)methylsiloxane homopolymer (HS—Si) was purchased from Gelest (SMS-992, MW range 4k-7k); silicone oil (Cat. No. 378364) was purchased from Sigma Aldrich, commercial pure S-nitroso-N-acetyl-D-penicillamine (SNAP) was purchase from Pharmablock (USA) Inc. (Hatfield, PA). Silicone rubber sheet (0.06 inches thick, Cat. No. 87315K13) was purchased from McMaster-Carr (USA). All materials were used without purification unless mentioned specifically. Luria Bertani (LB) broth media and agar were purchased from Sigma Aldrich. The bacterial strain Staphylococcus aureus (S. aureus, ATCC 6538) was obtained from American Type Culture Collection (ATCC).

Synthesis of S-nitrosothiol-based silicone oil (RSNO—Si). RSNO—Si was synthesized from (mercaptopropyl)methylsiloxane homopolymer (Si—SH) by nitrosation using clean t-butyl nitrite. Clean t-butyl nitrite was prepared by mixing an equal volume of 20 mM cyclam and t-butyl nitrite vigorously to chelate trace metal, and the procedure was repeated three times 31. The Si—SH (1 g) was dissolved in THF (10 mL), and clean t-butyl nitrite was added to the solution to nitrosate the free thiol and form the RSNO—Si. RSNO—Si with different RSNO contents were synthesized. The Si—SH (1 g, 1 equiv.) and THF (2 mL) were mixed in a scintillation vial, then 14.5 μL, 73 μL, 145 μL, and 290 μL of clean t-butyl nitrite (equivalent to 0.1%, 0.5%, 1% and 2% of thiols) were added to the solution to make RSNO0.1—Si, RSNO0.5—Si, RSNO1-Si, and RSNO2-Si, respectively. [The x value in the RSNOx-Si indicates the t-butyl nitrite addition resulting in x % of thiols in this reaction.] The solutions were stirred for 1 h, and the color changed from transparent to red, suggesting the formation of RSNO—Si. After the reaction, THF was removed by rotary evaporation, and the red RSNO—Si oils (RSNO0.1—Si, RSNO0.5—Si, RSNO1-Si, and RSNO2-Si) were obtained. These RSNO—Si oils were prepared fresh stored in the −20° C. freezer until further characterization.

NMR characterization of RSNO—Si oils. RSNO1-Si was chosen to be the representative sample for NMR analysis to track the changes during synthesis. RSNO1-Si (25 mg) was dissolved in CDCl₃ (600 μL), and characterized by a Varian/Agilent VNMRS 600 MHz with a 5 mm HCN cold probe and cooled carbon preamp. ¹H and ¹³C NMR were reported in ppm relative to the internal solvent resonances of CDCl₃, with 64 and 216 scans, respectively.

FT-IR characterization of RNSO—Si oils. Fourier transform infrared spectroscopy (FT-IR) was used to characterize the RSNO—Si structures. FT-IR spectra were taken by a PerkinElmer FT-IR Spectrum 3 spectrometer with KBr pellet. A drop of the RSNO—Si oil sample was added to the KBr pellet and gently wiped by Kimwipe until a thin layer of oil was left on the surface. The FT-IR measurement was obtained at 2 cm⁻¹ resolution and 32 scans over the wavenumber range of 500-4000 cm⁻¹.

Payload of RSNO—Si. The RSNO payload of RSNO—Si oils was determined with a Cary 60 UV-vis spectrophotometer. The RSNO—Si samples were dissolved in THE at 1 mg mL⁻¹ concentration and characterized by a Cary 60 UV-vis spectrometer at 300-500 nm with a medium scan speed. A calibration curve of RSNO functional group in THF using commercial pure S-nitroso-N-acetyl-D-penicillamine (SNAP) was made, and the RSNO—Si absorption at 340 nm was used to calculate RSNO payload based on this RSNO functional group calibration curve.

Viscosity of RSNO—Si. The viscosity of the series of RSNO—Si oils was measured by a Brookfield Viscometer (DV-II+Pro, Brookfield Ametek, USA), equipped with a CPE-40 spindle cone and sample cup. Water circulation was used to maintain the equipment temperature at rt (23° C.). In the viscometer sample cup, 0.5 mL of RSNO—Si oil was added, then the sample cup was placed back in the viscometer to start the test. The speed ramp was set up from 1 to 100 rpm with 10 increments, and each speed was held for 20 s with data recorded in each second. Each RSNO—Si oil was tested three times and the viscosity was averaged from three tests.

Preparation of RSNO—Si—SR disks. Medical-grade silicone rubber (SR) sheet was punched into disks with a diameter of 0.55 cm, then the SR disks were soaked in RSNO—Si oils at room temperature (rt) or −20° C. At the designed time point, disks were taken out and gently wiped by Kimwipe, and the weight of each disk was measured by a Mettler Toledo Excellence XSR analytical balance. The obtained weight of each disk after soaking (wₜ) was compared to the corresponding initial weight (Wo) to calculate the swelling ratio.

$$Swelling ratio ( \% ) = \frac{{w}_{t} - {w}_{0}}{{w}_{0}} \times 100 \%$$

Contact angles of RSNO—Si—SR disks. Static contact angles of samples were measured by an Ossila Contact Angle Goniometer (Ossila, UK). RSNO—Si—SR disks were placed on the sample stage of the instrument, and 5 μL of deionized water was dropped on the surface. The static contact angles were measured from still frames using the sessile drop approximation, and the results were analyzed by Ossila Contact Angle Software. Three individual water droplets were placed at 3 different locations on the surface and then averaged to get the average contact angles.

NO released from RSNO—Si—SR disks and leaching during NO release. NO released from the RSNO—Si—SR disk samples was analyzed by a Zysense chemiluminescence Nitric Oxide Analyzer (NOA) 280i as previously reported 32, and the nitrogen flow rate and cell pressure were set up at 200 mL min⁻¹ and 8.8-9.5 psi, respectively. PBS buffer (3 mL, 10 mM, pH 7.4) containing 100 μM EDTA was added to the sample vessel, and the sample vessel was partially submerged in a 37° C. water bath to mimic physiological temperature. Each test started with a short period of baseline measurement with only PBS buffer, then RSNO—Si—SR sample disk was placed in the buffer within the sample vessel. The NO released from the sample was purged by continuous N₂ flow and was detected in real-time by the chemiluminescence detector at 1 s intervals until it reached a steady-state. The NOA data was normalized with the surface area of samples to obtain the flux values with unit of mole cm⁻² min⁻¹. The NO release was quantified at various time points during the experiment to track the release trends, and samples were incubated in PBS at 37° C. between each measurement.

In this project, the potential leaching of RSNO—Si was obtained by soaking RSNO—Si—SR disks in 1 mL of 10 mM PBS (pH 7.4) with 100 μM EDTA in 37° C. incubator. Then, the soaking solution was checked by UV-vis to determine the amount of NO donor leached out. The sample vial holding RSNO—Si—SR disk was replenished with 1 mL of fresh 10 mM PBS (pH 7.4, 100 μM EDTA), and placed back to incubator until the next reading.

Antibacterial effects of RSNO—Si composite disks. To evaluate the antibacterial ability of RSNO—Si—SR samples (RSNO0.1—Si—SR, RSNO0.5—Si—SR and RSNO1-Si—SR), HS—Si—SR control, and unmodified SR controls were tested against S. aureus bacteria using a 4 h bacterial adhesion assay. For this, an isolated colony of S. aureus bacteria was inoculated and grown in LB media for 5 h at 120 rpm 37° C. The growth of bacteria in suspension was analyzed by recording the optical density (OD) of bacteria at 600 nm wavelength using UV-vis spectroscopy. Once the bacteria reached the mid-log phase, cells were extracted, washed, and re-suspended in sterile PBS. The OD₆₀₀ of bacteria was adjusted to ˜10⁷ CFU mL⁻¹ and exposed to UV sterilized samples (1 mL). Samples in bacterial suspension were incubated under constant agitation for 4 h at 120 rpm, 37° C. After 4 h of incubation, samples were rinsed with PBS to remove any loosely adhered cells on the surface and transferred into a new tube with fresh PBS. To extract the adhered cells, disks were homogenized and vortexed for 1 min each. Bacteria in the suspension were plated onto an LB agar plate using Bacteria Spiral Plater (Eddy Jet 2, IUL Instruments). After overnight incubation, viable bacterial colonies on the plates were enumerated using the plate counting method with SphereFlash Bacteria Colony Counter (IUL Instruments). Results from the antibacterial activity of the films are reported as a percent of viable colony forming units (CFU) on the surface of test films (RSNO—Si—SR and SH—Si—SR) vs. control (SR) normalized with the surface area of disks (CFU cm⁻²) (Equation 1).

$$\% Bacterial reduction = \frac{{C}_{SR} - {C}_{Test}}{{C}_{SR}} \times 100 \qquad ( Equation 1 )$$

Statistics. All the data were obtained with sample size ≥3, and data are reported as mean±standard deviation. A two-tailed Student's t-test with a hypothesis of unequal variance and α=0.05 were used to determine statistical significance.

### Results and Discussion

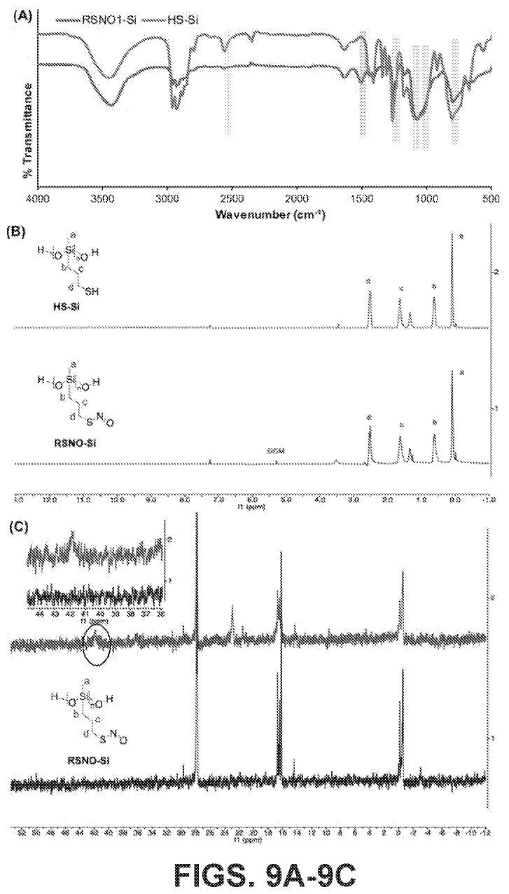

Preparation of S-nitrosothiol-based silicone oil (RSNO—Si). The synthesis of RSNO—Si oil was achieved by one-step nitrosation with the designed equivalence of clean t-butyl nitrite (FIG. 8). Once the reaction started, the transparent starting HS—Si oil quickly changed to a red color, which is the characteristic color of primary S-nitrosothiol (RSNO) functional group ³³, marking the formation of RSNO—Si oils. In addition to the visualization of color change, the RSNO group formation was also confirmed by FT-IR and NMR, using RSNO1-Si as a representative sample. As shown in FIG. 9A, both the HS—Si and RSNO1-Si showed the silicone oil characteristic peaks around 1260, 1090, and 800 cm⁻¹, and C—H stretching vibration peak at ˜ 2928 cm^{-1 34}. The sharp peak at 2558 cm⁻¹ was observed in both HS—Si and RSNO1-Si oils, indicating the S—H stretching ³⁵. Comparing the spectra of Si—H and RSNO1-Si, a peak ˜1500 cm⁻¹ appeared in the RSNO1-Si, which was assigned to the N—O stretching in the S-nitrosothiol structure ³⁵ and demonstrated the RSNO group formation in RSNO1-Si oil. However, a strong-SH peak was observed in RSNO1-Si sample, as 1% of thiols were converted to S-nitrosothiols, and many unreacted thiols remained in the structure.

NMR was also used to track RSNO functional group formation and confirm the successful synthesis of RSNO1-Si (FIG. 9B). While 1H NMR was not able to detect the structural changes in this nitrosation, ¹³C NMR of RSNO1-Si showed a chemical shift at 41.9 ppm, which was assigned to the carbon next to the S-nitrosothiol group. In HS—Si, this carbon is next to thiol and shows a chemical shift at 27.8 ppm ³⁶, but as the thiol is converted to RSNO, the chemical shift will move downfield. Usually, the ¹³C NMR of a carbons which are linked to the thiol groups are shifted downfield after the nitrosation step. The a carbons are shifted by 7-10 ppm for primary and secondary compounds, and shifted downfield by 10⁻²⁰ ppm for tertiary compounds ³⁷. In this case, a downfield chemical shift at 41.9 ppm appeared, which could be induced by the nitrosation. As mentioned in FT-IR, free thiols still existed in the structure, therefore, peaks related to HS—Si were observed in RSNO1-Si spectra.

The visualization of color changes in combination with the FT-IR and NMR results all demonstrated the RSNO1-Si formation, which indicates the covalent binding of NO to the free thiols present on the liquid silicone oil structure. In addition, the remaining free thiols available in the silicone oil structures may provide additional modification sites for the silicone oil. However, in this study, we only focused on RSNO—Si oils with the conversion from 0.1% (RSNO0.1—Si) to 2% (RSNO2-Si) to explore the reaction and tunable NO release and applications of RSNO—Si oils.

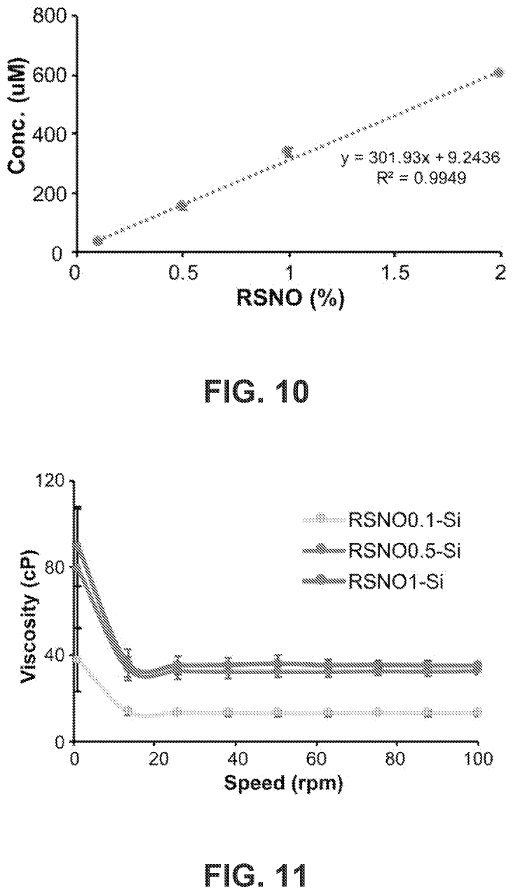

NO payloads of the RSNO—Si. The S-nitrosothiol group (RSNO) payload of RSNO—Si oils was determined by dissolving RSNO—Si in THF at 1 mg mL⁻¹, and calculated by absorption at 340 nm with a calibration curve. As shown in FIG. 10, RSNO0.1—Si, RSNO0.5—Si, RSNO1-Si, and RSNO2-Si had the NO payloads of 34.0+4.4 μM, 150.0+7.2 μM, 337.4 +14.8 μM, and 603.9+0.3 μM, respectively. The concentration of RSNO in RSNO—Si oils was found to be proportional to the initial clean t-butyl nitrite addition (e.g. 0.1%, 0.5%, 1%, and 2% to initial thiols). This result suggested that the accurate control of RSNO payload in RSNO—Si oils could be achieved by adjusting the addition of reactant clean t-butyl nitrite, and more reactant results in more RSNO payload.

Viscosity of RSNO—Si. The viscosity of RSNO—Si oils was evaluated to explore their physical property changes due to different payloads of RSNO. As shown in FIG. 11, at the 40-100 rpm range, RSNO0.1—Si, RSNO0.5—Si, and RSNO1-Si had a viscosity of 12.8+0.1 cP, 32.0+0.2 cP, and 35.1+0.3 cP, respectively. From these viscosity results, the more RSNO payload in the silicone oil, the more viscous the silicone oil will be as a result of disulfide formation that occurs over time as the RSNOs release their NO payload. Theoretically, the viscosity of RSNO—Si oils can be controlled by manipulating the RSNO payloads using the additional free thiols present on the silicone oil structure. However, further increasing the RSNO payload immobilized to the silicone oils results in further increased viscosity which over time solidifies the material. The trend of increasing viscosity with increased RSNO immobilization is due to RSNO decomposition mechanism which results resulting in disulfide formation, so maintaining the NO payload to the range studied herein will be beneficial.

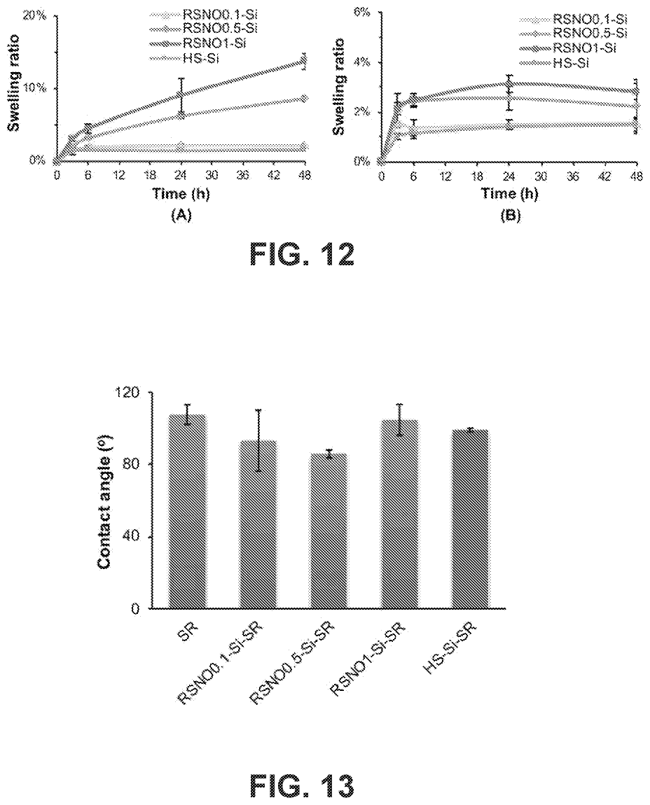

Swelling ratio of RSNO—Si—SR disks. In the remaining studies, the RSNO—Si oils were used to as a lubricant on commercial rubber disks (SR) via a thin layer of the RSNO—Si oils on the surface by infusing the disks in RSNO—Si oils. Because RSNO2-Si became viscous, only RSNO0.1—Si, RSNO0.5—Si, and RSNO1-Si were used for the further experiments. SR disks were soaked in RSNO—Si oils at either rt or −20° C., and the swelling ratio of each temperature was plotted in FIG. 12. The starting silicone oil, HS—Si, was also used to prepare control disks (HS—Si—SR) for the following experiments to understand how RSNO changed the silicone oil property and application. As shown in FIG. 12, the swelling ratio of SR disks in different silicone oils reached the steady-state in about 6 hours at −20° C. The RSNO1-Si—SR, RSNO0.5—Si—SR, RSNO0.1—Si—SR, and HS—Si—SR gained about 2.5 wt %, 2.4 wt %, 1.4 wt %, and 1.2 wt % of the corresponding oils on the disks. The different swelling ratios of the RSNO—Si—SR may relate to the different molecular weights and viscosities, as the RSNO1-Si—SR gained the most weight, while HS—Si—SR gained the least weight. At rt, RSNO0.1—Si—SR and HS—Si—SR reached a steady-state infusion around 6 h, but the RSNO0.5—Si—SR and RSNO1-Si—SR kept increasing, and the dramatic increments were likely due to the disulfide bond formation and crosslinking as the results of the decomposition of RSNO—Si at rt. Therefore, SR disks would be infused with the RSNO—Si oils at −20° C. freezer for 6 h to prepare sample disks for further characterizations.

Contact angle and sliding angle of RSNO—Si composite disks. The contact angles of RSNO—Si—SR disks were measured by Ossila Contact Angle Goniometer, and the results are shown FIG. 13. The original SR disks had contact angles 108+5°, indicating the surfaces were relatively hydrophobic; however, after silicone oil treatment, the contact angles decreased. The HS—Si—SR, RSNO0.1—Si—SR, RSNO0.5—Si—SR, and RSNO1-Si—SR had contact angles of 99+1°, 93+17°, 86+2°, and 105+9°, respectively. Considering the thiol groups on the HS—Si and RSNO—Si oils are hydrophilic, the infusion of these oils on SR surface provided slightly more hydrophilicity to the surface and decreased the contact angles. However, no significant trend of contact angle changes was observed among HS—Si and RSNO—Si oils, which may relate to the relatively low RSNO payload compared to the free thiols present on the silicone oil structure. For RSNO—Si oils in this study, only 0.1% to 1% of the HS groups were converted to RSNO groups, so the hydrophilicity of original HS—Si and the RSNO—Si oils could be slightly different as observed in this study.

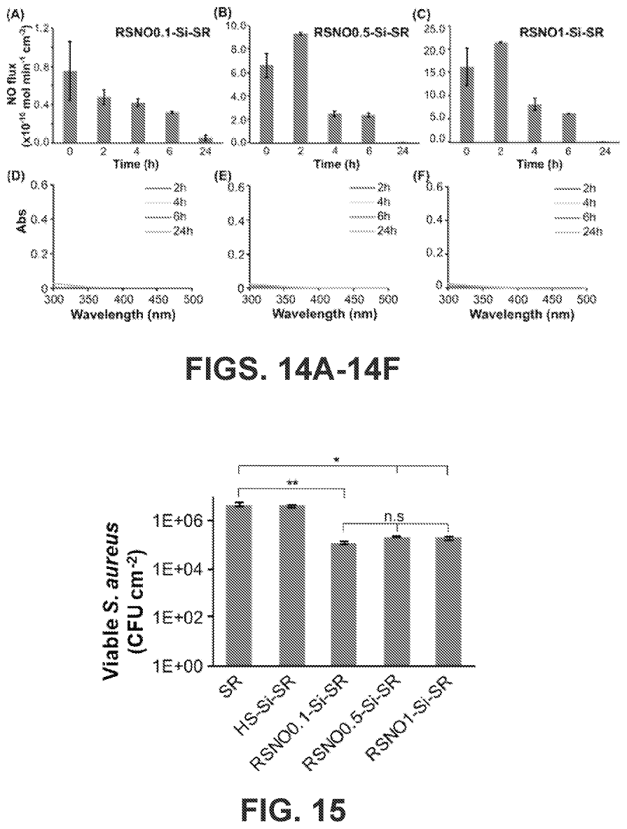

NO released from RSNO—Si composite disks and the leaching during release. The NO released from the RSNO—Si—SR disks was measured by a gold standard chemiluminescence method using a Zysense chemiluminescence NOA 280i as reported^{28, 32, 38}. As shown in FIG. 14A-C, all RSNO—Si—SR disks released NO for 24 h at different flux levels. During the initial 4 h of NO release characterization, the least payload RSNO0.1—Si—SR released NO at 0.4-0.8×10⁻¹⁰ mole cm⁻² min⁻¹, the medium payload RSNO0.5—Si—SR released NO at 2.5-9.3×10⁻¹⁰ mole cm⁻² min⁻¹, while the most payload RSNO1-Si—SR release NO at 8.1-21.5×10⁻¹⁰ mole cm⁻² min⁻¹. All samples released NO around or above 0.5×10⁻¹⁰ mole cm⁻² min⁻¹, which is considered to be the minimum physiological level of NO³⁹. Clearly, the increasing the RSNO payload in the oil results in the higher NO release that was observed. The RSNO—Si oils demonstrated this trend because the release of NO from the RSNO moiety follows the first-order kinetics which directly points to concentration. The RSNO1-Si has the highest concentration of the RSNO group (1 wt % RSNO, 337.4+14.8 μM) among RSNO0.1—Si to RSNO1-Si; therefore, it should have the fastest reaction rate and release NO at the highest level. The relationship between NO release levels and RSNO payloads also revealed a possibility to tune the NO release for different applications which require different NO flux levels, by simply adjusting the NO payload at the synthesis step. The RSNO0.5—Si and RSNO1-Si reached the maximum NO flux levels at 2 h while RSNO0.1—Si reached the maximum flux level at 0 h. The different maxima timepoint could attribute to the different concentrations of RSNO payloads and the first order NO release mechanism. Compared to the RSNO0.1—Si, the higher RSNO conversions in the RSNO0.5—Si and RSNO1-Si had more payloads which would decompose within a longer initiation period and generate more thiol radicals, which then catalyze the RSNO decomposition and result in increased NO release levels.

The leaching behavior is another issue that needs to be monitored because NO donor leaching from biomaterials may cause a delocalized release of NO in the system. Therefore, the leaching of the RSNO—Si during the NO release was characterized by UV-vis spectroscopy (FIG. 14C-14E). The characteristic peak of the RSNO moiety is at ˜340 nm. However, in this case, at 340 nm no peak was detected after the RSNO—Si—SR sample had incubated in the buffer in a 24 h period, indicating that no RSNO—Si was leached out of the RSNO—Si—SR disks. The RSNO moiety is covalently linked to the hydrophobic silicone oil structure, and the RSNO—Si oils were not soluble in water. Based on the NO release profiles and the leaching assay results, the RSNO—Si oils can be tuned to control the NO release from biomaterial interfaces without leaching. These properties could be great advantages for applications which require different levels of NO.

Antibacterial effects of RSNO—Si composite disks. The ability of RSNO—Si—SR disks to inhibit the attachment of bacteria and subsequent biofilm formation was tested against S. aureus bacteria in a 4 h bacterial adhesion assay. Medical devices made from silicone rubber polymers such as urinary, vascular catheters, catheters, and cannulas possess suitable surfaces for the bacteria to attach, proliferate, and develop a biofilm. Opportunistic pathogens like S. aureus can use these devices as a medium to invade the body leading to severe bodily infections.⁴⁰ These issues negatively impact the durability of the device and as well as prove life-threatening for patients especially those that are immunocompromised. Therefore, the development of antibacterial strategies to prevent the initial attachment of bacteria on the surface is highly desired to prevent infection in long-term biomedical device applications. NO-releasing materials have been proven to reduce the incidences of bacterial contamination on the surfaces providing an efficient way to combat device-associated infections ⁴¹⁻⁴³.

The results from the study revealed that the RSNO0.1—Si—SR, RSNO0.5—Si—SR, and RSNO1-Si—SR disks resulted in 97.45, 95.40, and 96.08% reduction of S. aureus bacteria on the surface as compared to the SR control, respectively (p<0.05) (FIG. 15). No significant difference was observed among RSNO0.1—Si—SR, RSNO0.5—Si—SR and RSNO1-Si—SR disks. While the SH—Si—SR control also resulted in bacterial reduction (18.47%), the greatest reduction was observed with the RSNO—Si—SR samples due to the active release of NO from the surfaces. Previous studies have demonstrated a similar reduction in bacteria with oil control due to the introduction of slippery surfaces via infusion of silicone oil. ⁴⁴ The one-step RSNO conjugated silicone oil infusion has an advantage over the prior reports of a two-step infusion process. The covalent binding of oil to NO donor helps in the premature loss of donor preventing leaching and its potential side effects.

The results presented herein demonstrate a simple method to synthesize NO-releasing liquid silicone oils that have many possible future applications in biomaterials, medical devices, and potentially other areas. The studies conducted here demonstrate the ability of these NO-releasing silicone oils to be used as a lubricant on polymeric biomaterial surfaces, and this application imparts antimicrobial properties at that material interface which is advantageous for many applications. However, future studies will need to further evaluate this approach of functionalizing silicone oils with NO-releasing moieties. For example, various NO-releasing moieties (e.g., primary or tertiary RSNOs) could be covalently immobilized to the silicone oils and the effects on the NO release properties assessed. Also, storage stability studies can elucidate how stable various NO-releasing moieties are when immobilized to silicone oils, which would be a critical factor in real-world applications where these materials could be stored either at room temperature or at refrigerated conditions. These studies are the critical next steps necessary to develop optimized NO-releasing silicone oil materials and facilitate the translation of these materials to their intended antimicrobial applications.

### Conclusions

In this study, the RSNO—Si oil was successfully synthesized by coupling the primary S-nitrosothiol donor to silicone oil, and the products were confirmed by FT-IR, ¹H and ¹³C NMR. The RSNO0.1—Si, RSNO0.5—Si, RSNO1-Si, and RSNO2-Si had the NO payloads of 34.0±4.4 μM, 150.0±7.2 μM, 337.4±14.8 μM, and 603.9±0.3 μM, respectively, suggesting the NO payloads could be tuned by the reaction stoichiometry. In addition, the NO payload affected the viscosity of RSNO—Si oils and the swelling ratio of SR disks in the RSNO—Si oils. The RSNO0.1—Si, RSNO0.5—Si, and RSNO1-Si had a viscosity of 12.8±0.1 cP, 32.0±0.2 cP, and 35.1±0.3 cP, respectively, suggesting that the increased NO payloads resulted to higher viscosity. The swelling ratios of SR disks in RSNO—Si oils were also related to viscosity, and SR treated with RSNO oil with lower NO payload had higher swelling ratio at both rt and−20° C. Even though RSNO—Si oils with different NO payloads did not show differences at contact angles, their NO release profiles were very different. All sample disks were able to release NO for 24 h without NO donor leaching. In the first 4 h, RSNO0.1—Si—SR, RSNO0.5—Si—SR, and RSNO1-Si—SR released NO at 0.4-0.8×10⁻¹⁰ mole cm⁻² min⁻¹, 2.5-6.5×10⁻¹⁰ mole cm⁻² min⁻¹, and 8.1-21.5×10⁻¹⁰ mole cm⁻² min⁻¹, respectively. Due to the NO-releasing RSNO—Si interfaces, the RSNO—Si—SR disks could inhibit the attachment of viable S. aureus bacteria. The RSNO0.1—Si—SR, RSNO0.5—Si—SR, and RSNO1-Si—SR disks were exhibited a 97.45, 95.40, and 96.08% reduction of S. aureus, respectively, on the surface as compared to SR control in a 4 h bacterial adhesion assay (p<0.05). The novel RSNO—Si oils demonstrated their easy synthesis with tunable NO payload, suitable viscosity for application, tunable range of NO release flux levels without leaching, as well as excellent inhibition effects of S. aureus attachment on SR surfaces. Due to these advantages of RSNO—Si oils, they could be used to fabricate the NO-releasing antibacterial interface on medical devices and be utilized as a new platform material to deliver therapeutic NO at different levels.

### REFERENCES FOR EXAMPLE 2

1. VanEpps, J. S.; Younger, J. G., Implantable Device-Related Infection. Shock 2016, 46 (6), 597-608.

2. Rabih, Device-Associated Infections: A Macroproblem That Starts with Microadherence. Clin. Infect. Dis. 2001, 33 (9), 1567-1572.

3. Guinn, T. S.; Bailey, G. J.; Mecklenburg, R. S., Factors Related to Discontinuation of Continuous Subcutaneous Insulin-Infusion Therapy. Diabetes Care 1988, 11 (1), 46-51.

4. Mecklenburg, R. S.; Benson, E. A.; Benson, J. W., Jr.; Fredlund, P. N.; Guinn, T.; Metz, R. J.; Nielsen, R. L.; Sanner, C. A., Acute Complications Associated with Insulin Infusion Pump Therapy. Report of Experience with 161 Patients. JAMA 1984, 252 (23), 3265-9.

5. Guenego, A.; Bouzille, G.; Breitel, S.; Esvant, A.; Poirier, J.-Y.; Bonnet, F.; Guilhem, I., Insulin Pump Failures: Has There Been an Improvement? Update of a Prospective Observational Study. Diabetes Technology and Therapeutics 2016, 18 (12), 820-824.

6. Jarosz-Chobot, P.; Nowakowska, M.; Polanska, J., Seeking the Factors Predisposing to Local Skin Inflammatory State Development in Children with Type 1 Diabetes (T1dm) Treated with Continuous Subcutaneous Insulin Infusion (Csii). Experimental and Clinical Endocrinology & Diabetes 2007, 115 (03), 179-181.

7. Nowakowska, M.; Jarosz-Chobot, P.; Polańska, J., Bacterial Strains Colonizing Subcutaneous Catheters of Personal Insulin Pumps. Polish journal of microbiology 2007, 56 (4), 239-243.

8. Al Hayek, A. A.; Robert, A. A.; Al Dawish, M. A., Skin-Related Complications among Adolescents with Type 1 Diabetes Using Insulin Pump Therapy. Clin Med Insights Endocrinol Diabetes 2018, 11, 1179551418798794.

9. Verderosa, A. D.; Totsika, M.; Fairfull-Smith, K. E., Bacterial Biofilm Eradication Agents: A Current Review. Frontiers in Chemistry 2019, 7, 824.

10. Schairer, D. O.; Chouake, J. S.; Nosanchuk, J. D.; Friedman, A. J., The Potential of Nitric Oxide Releasing Therapies as Antimicrobial Agents. Virulence 2012, 3 (3), 271-279.

11. Qian, Y.; Matson, J. B., Gasotransmitter Delivery Via Self-Assembling Peptides: Treating Diseases with Natural Signaling Gases. Adv. Drug Deliv. Rev. 2016, 110-111, 137-156.

12. Garren, M. R.; Ashcraft, M.; Qian, Y.; Douglass, M.; Brisbois, E. J.; Handa, H., Nitric Oxide and Viral Infection: Recent Developments in Antiviral Therapies and Platforms. Applied Materials Today 2021, 22, 100887.

13. Carpenter, A. W.; Schoenfisch, M. H., Nitric Oxide Release: Part li. Therapeutic Applications. Chem. Soc. Rev. 2012, 41 (10), 3742-3752.

14. Lisi, F.; Zelikin, A. N.; Chandrawati, R., Nitric Oxide to Fight Viral Infections. Advanced Science 2021, 8 (7), 2003895.

15. González Calderón, J. A.; Contreras López, D.; Pérez, E.; Vallejo Montesinos, J., Polysiloxanes as Polymer Matrices in Biomedical Engineering: Their Interesting Properties as the Reason for the Use in Medical Sciences. Polym. Bull. 2020, 77 (5), 2749-2817.

16. Mata, A.; Fleischman, A. J.; Roy, S., Characterization of Polydimethylsiloxane (Pdms) Properties for Biomedical Micro/Nanosystems. Biomed. Microdevices 2005, 7 (4), 281-293.

17. El-Zaim, H. S.; Heggers, J. P., Silicones for Pharmaceutical and Biomedical Applications. 2nd Edition ed.; Taylor & Francis Group: 2001; p 79-88.

18. Dimitrova, M. N.; Bee, J. S.; Lu, L.; Fernandez, J. E., Development of Prefilled Syringe Combination Products for Biologics. Springer International Publishing: 2018, pp 203-224.

19. Yuan, W.; Hu, Z.; Liao, M.; Cai, Y.; Lele, M.; Liu, Z.; Chen, Y.; Liu, Y.; Lu, N., A Novel Preparation Method for Silicone Oil Nanoemulsions and Its Application for Coating Hair with Silicone. Int. J. Nanomedicine 2012, 5719.

20. Aziz, T.; Fan, H.; Khan, F. U.; Haroon, M.; Cheng, L., Modified Silicone Oil Types, Mechanical Properties and Applications. Polym. Bull. 2019, 76 (4), 2129-2145.

21. Crowe, F. W., Use of Silicone Oils in Maintaining Surgical Instruments. Journal of the American Medical Association 1952, 149 (16), 1464.

22. Kasapgil, E.; Badv, M.; Cantú, C. A.; Rahmani, S.; Erbil, H. Y.; Anac Sakir, I.; Weitz, J. I.; Hosseini-Doust, Z.; Didar, T. F., Polysiloxane Nanofilaments Infused with Silicone Oil Prevent Bacterial Adhesion and Suppress Thrombosis on Intranasal Splints. ACS Biomater. Sci. Eng. 2021, 7 (2), 541-552.

23. Gonçalves, A. C.; Cavassana, S.; Chavarette, F. R.; Outa, R.; Casarin, S. J.; Corazza, A. V., Variation of the Penetration Effort in an Artificial Tissue by Hypodermic Needles. Journal of Healthcare Engineering 2020, 2020, 8822686.

24. Lavielle, N.; Asker, D.; Hatton, B. D., Lubrication Dynamics of Swollen Silicones to Limit Long Term Fouling and Microbial Biofilms. Soft Matter 2021, 17 (4), 936-946.

25. Krayukhina, E.; Tsumoto, K.; Uchiyama, S.; Fukui, K., Effects of Syringe Material and Silicone Oil Lubrication on the Stability of Pharmaceutical Proteins. Journal of Pharmaceutical Sciences 2015, 104 (2), 527-35.

26. MacCallum, N.; Howell, C.; Kim, P.; Sun, D.; Friedlander, R.; Ranisau, J.; Ahanotu, O.; Lin, J. J.; Vena, A.; Hatton, B.; Wong, T.-S.; Aizenberg, J., Liquid-Infused Silicone as a Biofouling-Free Medical Material. ACS Biomater. Sci. Eng. 2014, 1 (1), 43-51.

27. Goudie, M. J.; Pant, J.; Handa, H., Liquid-Infused Nitric Oxide-Releasing (Linorel) Silicone for Decreased Fouling, Thrombosis, and Infection of Medical Devices. Sci. Rep. 2017, 7 (1), 13623.

28. Chug, M. K.; Feit, C.; Brisbois, E. J., Increasing the Lifetime of Insulin Cannula with Antifouling and Nitric Oxide Releasing Properties. ACS Appl. Bio Mater. 2019, 2 (12), 5965-5975.

29. Douglass, M.; Hopkins, S.; Chug, M. K.; Kim, G.; Garren, M. R.; Ashcraft, M.; Nguyen, D. T.; Tayag, N.; Handa, H.; Brisbois, E. J., Reduction in Foreign Body Response and Improved Antimicrobial Efficacy Via Silicone-Oil-Infused Nitric-Oxide-Releasing Medical-Grade Cannulas. ACS Appl. Mater. Interfaces 2021, 13 (44), 52425-52434.

30. Homeyer, K. H.; Goudie, M. J.; Singha, P.; Handa, H., Liquid-Infused Nitric-Oxide-Releasing Silicone Foley Urinary Catheters for Prevention of Catheter-Associated Urinary Tract Infections. ACS Biomater. Sci. Eng. 2019, 5 (4), 2021-2029.

31. Goudie, M. J.; Singha, P.; Hopkins, S. P.; Brisbois, E. J.; Handa, H., Active Release of an Antimicrobial and Antiplatelet Agent from a Nonfouling Surface Modification. ACS Appl. Mater. Interfaces 2019, 11 (4), 4523-4530.

32. Feit, C. G.; Chug, M. K.; Brisbois, E. J., Development of S-Nitroso-N-Acetylpenicillamine Impregnated Medical Grade Polyvinyl Chloride for Antimicrobial Medical Device Interfaces. ACS Appl. Bio Mater. 2019, 2 (10), 4335-4345.

33. Zhang, C.; Biggs, T. D.; Devarie-Baez, N. O.; Shuang, S.; Dong, C.; Xian, M., S-Nitrosothiols: Chemistry and Reactions. Chem. Commun. 2017, 53 (82), 11266-11277.

34. Chen, D.; Hu, X.; Zhang, H.; Yin, X.; Zhou, Y., Preparation and Properties of Novel Polydimethylsiloxane Composites Using Polyvinylsilsesquioxanes as Reinforcing Agent. Polymer Degradation and Stability 2015, 111, 124-130.

35. Arulsamy, N.; Bohle, D. S.; Butt, J. A.; Irvine, G. J.; Jordan, P. A.; Sagan, E., Interrelationships between Conformational Dynamics and the Redox Chemistry Ofs-Nitrosothiols. J. Am. Chem. Soc. 1999, 121 (30), 7115-7123.

36. Karuppasamy, K.; Prasanna, K.; Vikraman, D.; Kim, H.-S.; Kathalingam, A.; Mitu, L.; Rhee, H., A Rapid One-Pot Synthesis of Novel High-Purity Methacrylic Phosphonic Acid (Pa)-Based Polyhedral Oligomeric Silsesquioxane (Poss) Frameworks Via Thiol-Ene Click Reaction. Polymers-Basel 2017, 9 (12), 192.

37. Szaciłowski, K.; Stasicka, Z., S-Nitrosothiols: Materials, Reactivity and Mechanisms. Progress in Reaction Kinetics and Mechanism 2001, 26 (1), 1-58.

38. Chug, M. K.; Bachtiar, E.; Narwold, N.; Gall, K.; Brisbois, E. J., Tailoring Nitric Oxide Release with Additive Manufacturing to Create Antimicrobial Surfaces. Biomater. Sci. 2021, 9 (8), 3100-3111.

39. Vaughn, M. W., Estimation of Nitric Oxide Production and Reaction Rates in Tissue by Use of a Mathematical Model. Am. J. Physiol. Heart Circ. Physiol. 1998, 274 (6), H2163-H2176.

40. Zheng, Y.; He, L.; Asiamah, T. K.; Otto, M., Colonization of Medical Devices by Staphylococci. Environmental Microbiology 2018, 20 (9), 3141-3153.

41. Brisbois, E. J.; Bayliss, J.; Wu, J.; Major, T. C.; Xi, C.; Wang, S. C.; Bartlett, R. H.; Handa, H.; Meyerhoff, M. E., Optimized Polymeric Film-Based Nitric Oxide Delivery Inhibits Bacterial Growth in a Mouse Burn Wound Model. Acta Biomater. 2014, 10 (10), 4136-4142.

42. Chug, M. K.; Bachtiar, E.; Narwold, N.; Gall, K.; Brisbois, E. J., Tailoring Nitric Oxide Release with Additive Manufacturing to Create Antimicrobial Surfaces. Biomater. Sci. 2021, 9 (8), 3100-3111.

43. Colletta, A.; Wu, J.; Wo, Y.; Kappler, M.; Chen, H.; Xi, C.; Meyerhoff, M. E., S-Nitroso-N-Acetylpenicillamine (Snap) Impregnated Silicone Foley Catheters: A Potential Biomaterial/Device to Prevent Catheter-Associated Urinary Tract Infections. ACS Biomater. Sci. Eng. 2015, 1 (6), 416-424.

44. Chug, M. K.; Feit, C.; Brisbois, E. J., Increasing the Lifetime of Insulin Cannula with Antifouling and Nitric Oxide Releasing Properties. ACS Applied Bio Materials 2019, 2 (12), 5965-5975.

### Example 3

### Materials and Methods

### Synthesis of S-nitroso-N-acetylpenicillamine-based silicone oil (SNAP-Si)

N-acetyl-D-penicillamine thioactone (NAP-thiolactone) was prepared first from N-acetyl-D-penicillamine (NAP), and SNAP-Si was synthesized by coupling N-acetyl-D-penicillamine thioactone (NAP-thiolactone) to poly [dimethylsiloxane-co-(3-aminopropyl)methylsiloxane] (NH₂—Si), followed by nitrosation. NH₂—Si (5 g, Si), NAP-thiolactone (1.2 g), and dichloromethane (50 mL, DCM) were placed in a 250 mL round bottom flask and reacted for 18 hours at room temperature (rt, 23° C.) to form NAP coupled silicone oil (NAP-Si). Clean t-butyl nitrite was prepared by washing t-butyl nitrite with equal volume of 20 mM cyclam solution three times. Then, clean t-butyl nitrite (0.82 mL) and DCM (2 mL) was added to the NAP-Si and stirred at rt for 30 min to form SNAP-Si. During the reaction, green color solution formed immediately, and the solution was condensed at 40° C. to remove excessive solvents. SNAP-Si was stored in−20° C. freezer for further experiments.

### Preparation of SNAP—Si Based Polymer Films

The SNAP—Si based polymer films were prepared by blending SNAP-Si oil with polymers (CarboSil® 20 80A, Tecoflex SG 80A, and Tecophilic SP-60D-60 polymers). Each polymer was dissolved into THE at 60 mg mL⁻¹ after stirring overnight, then 10 wt % of SNAP-Si oil was added to polymer solution, and the mixture was poured in a mold, and airdried slowly in a box in hood. Based on different polymer solutions, the formed SNAP—Si based polymer films were named as ssCB, ssTF, and ssTP for CarboSil®, Tecoflex SG 80A, and Tecophilic SP-60D-60, respectively.

NO Released from SNAP—Si Based Polymer Films and Leaching During NO release

NO released from the SNAP—Si based polymer films (ssCB, ssTF, ssTP) was analyzed by a Zysense chemiluminescence Nitric Oxide Analyzer (NOA) 280i as previously reported. The supply nitrogen flow rate was set up at 200 mL min⁻¹, and cell pressure was set up at 8.8-9.5 psi. SNAP—Si based polymer films were punched into small disks with diameters of 0.7 cm. PBS buffer (3 mL, 10 mM, pH 7.4) containing 100 μM EDTA was added to sample vessel, and the sample vessel was partially submerged in a 37° C. water bath to mimic physiological temperature. Each test started with a short baseline measurement, then a sample disk was placed in the buffer in the sample vessel. NO released from the sample under physiological temperature. NO was purged by continuous N₂ flow and was detected in real-time by the chemiluminescence detector in 1 s interval until it reached stead-state. The NOA data was normalized with the surface area of samples to obtain the flux values with unit of mole cm⁻² min⁻¹. NO release was quantified at various time points during the experiment to track the release trends, and samples were incubated in PBS at 37° C. between each measurement.

### Antibacterial Effects of SNAP—Si Based Carbosil® Polymer Disks

SNAP—Si based Carbosil® film (ssCB) was chosen to test he antibacterial effects against S. aureus via a 24 h bacterial adhesion assay. NAP-Si oil was blended with Carbosil® at 10 wt % to make NAP-CB film as negative control of ssCB, and CB film was tested as the control. An isolated colony of S. aureus bacteria was inoculated and grown in LB media for 5 h at 120 rpm 37° C. Growth of bacteria in suspension was analyzed by recording optical density (OD) of bacteria at 600 nm wavelength using UV-vis spectroscopy. Once the bacteria reached mid-log phase, cells were extracted, washed, and re-suspended in sterile PBS. The OD₆₀₀ of bacteria was adjusted to ˜10⁷ CFU mL⁻¹ and exposed to UV sterilized samples (1 mL). Samples in bacterial suspension were incubated under constant agitation for 24 h at 120 rpm, 37° C. After 24 h of incubation, samples were rinsed with PBS to remove any loosely adhered cells on the surface and transferred into a new tube with fresh PBS. To extract the adhered cells, disks were homogenized and vortexed for 1 min each. Bacteria in the suspension was plated onto an LB agar plate using Bacteria Spiral Plater (Eddy Jet 2, IUL Instruments). After overnight incubation, viable bacterial colonies on the plates were enumerated using plate counting method with SphereFlash Bacteria Colony Counter (IUL Instruments). Results from the antibacterial activity of the films are reported as a percent of viable colony forming units (CFU) on the surface of test films (R⁰.5C and HS—C vs. control (SR) normalized with the surface area of disks (CFU cm⁻²) (equation 1).

$$\% Bacterial reduction = \frac{{C}_{SR} - {C}_{Test}}{{C}_{SR}} \times 100 \qquad ( 1 )$$

### Results and Discussion

NO Released from SNAP—Si Based Polymer Films

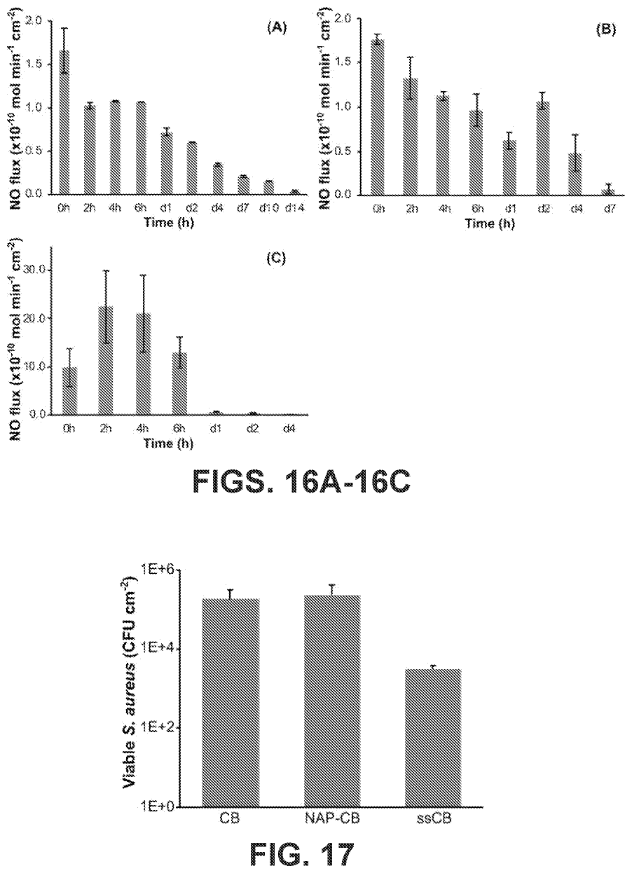

NO released from the RC films was measured by a gold standard chemiluminescence method using a Zysense chemiluminescence NOA 280i as reported. As shown in FIGS. 16A-16C, three films demonstrated different lengths of NO release period. ssCB released NO for 14 d, ssTF release NO for 7 d, and ssTP only release NO for 4 d. ssCB release NO at 1.66+0.26 ×10⁻¹⁰ mol min⁻¹ cm⁻² in the 1 h, and about 0.97-1.33 ×10⁻¹⁰ mol min⁻¹ cm⁻² for the following 6h. ssTF release NO at 1.77±0.06 ×10⁻¹⁰ mol min⁻¹ cm⁻² in the 1 h, and maintained about 1 ×10⁻¹⁰ mol min⁻¹ cm⁻² for the following 6h. ssTP release NO at 9.83 ±3.90×10⁻¹⁰ mol min⁻¹ cm⁻² in the 1 h, and maintained about 12.98-22.42 ×10⁻¹⁰ mol min⁻¹ cm-2 for the following 6 h. The NO release flux level of ssCB and ssTF were very similar, while ssTF were slightly higher than ssCB. On the contrary, ssTP was very different, and it release the highest level of NO among three groups of samples, and the NO release period was shortest. These phenomena may relate to the water uptake of polymers, which limits the water diffusion into polymer matrices. The NO release is regulated by the decomposition of SNAP donor and generate thiyl radicals to trigger more reaction and NO release. When polymer has high water uptake, water diffuses into the matrices easily, and increases the free volume and mobility of the molecular chains, which enhances the chance of thiyl radicals to trigger further reactions and form crosslinking.

The water uptake of ssCB, ssTF and ssTP were 2.27±0.08%, 1.77±0.08%, and 1.59±0.19%, respectively. For determining water uptake, samples of the polymer films were weighed and then immersed in PBS for 48 h at 37° C. The wet films were wiped dry and weighed again. The water uptake of the polymer films is reported in weight percent as follows: water uptake (wt %)=(Wwet−Wdry)/Wdry×100, where Wwet and Wdry are the weights of the wet and dry films, respectively (see Brisbois EJ, Handa H, Major TC, Bartlett RH, Meyerhoff ME. Long-term nitric oxide release and elevated temperature stability with S-nitroso-N-acetylpenicillamine (SNAP)-doped Elast-eon E2As polymer. Biomaterials. 2013; 34 (28): 6957-66; Brisbois EJ, Bayliss J, Wu J, Major TC, Xi C, Wang SC, Bartlett RH, Handa H, Meyerhoff ME. Optimized polymeric film-based nitric oxide delivery inhibits bacterial growth in a mouse burn wound model. Acta Biomaterialia. 2014; 10 (10): 4136-42).

ssTP has the most water adsorption, thus it is easier for water molecules to diffuse into the matrix and cause more NO release compared to less water uptake ssCB or SSTF. ssCB exhibited the least water uptake capability, and also demonstrated the longest NO release period as well as the lowest NO release flux. The results of water uptake and NO release flux and period matched each other, implying a new strategy to tune NO release of polymer films by choosing polymer matrices with different water uptakes.

Antibacterial Effects of ssCB Film

All NO-releasing polymers demonstrated NO release, and ssCB had the longest NO release period with low level of NO flux. Therefore, ssCB was chosen as the representative polymer to check the antibacterial effects of SNAP—Si based polymers. CB film was used as the control, and NAP-CB film was also prepared as a negative control. NAP-CB was formulated with NAP-Si, which has similar structure as SNAP-Si. The only difference between NAP-CB and ssCB was NAP-CB does not release NO.

The ability of ssCB film to inhibit the attachment of bacteria and subsequent biofilm formation was tested against S. aureus bacteria in a 24 h bacterial adhesion assay. Opportunistic pathogens like S. aureus can use these devices as a medium to invade the body leading to severe bodily infections, ¹ and these issues negatively impact the durability of the device and as well as prove life-threatening for patients especially those that are immunocompromised. Because of these problems, development of antibacterial strategies to prevent the initial attachment of bacteria on the surface are highly desired to stop the biofilm formation in long-term biomedical device applications. NO-releasing materials have been proven to reduce the incidences of bacterial contamination on the surfaces providing an efficient way to combat device associated infections. ²⁻⁴

The results of this assay revealed that ssCB films resulted in 98.23% reduction of S. aureus bacteria on the surface as compared to CB control, while NAP-CB increased 32.17% of bacterial on the surface compared to CB control (FIG. 17). No significant difference was observed among CB and NAP-CB films, while ssCB was significantly different from CB (p<0.1). Comparison of the NAP-CB and ssCB suggested that NO was the reason for different killing effects. Previous studies have demonstrated similar reduction in bacteria with NO-releasing silicone oils due to the antibacterial NO gas originated from the RSNO—Si additive in the polymer films, and ssCB was proved to be a potential antibacterial material as it prevents the leaching issue while providing long term NO release for up to 2 weeks.

### REFERENCES FOR EXAMPLE 3

1. Zheng, Y.; He, L.; Asiamah, T. K.; Otto, M., Colonization of Medical Devices by Staphylococci. Environmental Microbiology 2018, 20 (9), 3141-3153.

2. Brisbois, E. J.; Bayliss, J.; Wu, J.; Major, T. C.; Xi, C.; Wang, S. C.; Bartlett, R. H.; Handa, H.; Meyerhoff, M. E., Optimized Polymeric Film-Based Nitric Oxide Delivery Inhibits Bacterial Growth in a Mouse Burn Wound Model. Acta Biomater. 2014, 10 (10), 4136-4142.

3. Chug, M. K.; Bachtiar, E.; Narwold, N.; Gall, K.; Brisbois, E. J., Tailoring Nitric Oxide Release with Additive Manufacturing to Create Antimicrobial Surfaces. Biomater. Sci. 2021.

4. Colletta, A.; Wu, J.; Wo, Y.; Kappler, M.; Chen, H.; Xi, C.; Meyerhoff, M. E., S-Nitroso-N-Acetylpenicillamine (Snap) Impregnated Silicone Foley Catheters: A Potential Biomaterial/Device to Prevent Catheter-Associated Urinary Tract Infections. ACS Biomater. Sci. Eng. 2015, 1 (6), 416-424.

1. A nitric oxide releasing polysiloxane comprising a polysiloxane backbone with one or more nitric oxide releasing moieties pendant to the polysiloxane backbone.

2. The nitric oxide releasing polysiloxane of claim 1, wherein the polysiloxane backbone comprises a dialkyl polysiloxane.

3. The nitric oxide releasing polysiloxane of claim 1, wherein the polysiloxane comprises one or more units having the structure I 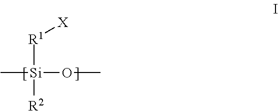
wherein R¹ is a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy;
R² is an alkyl group; and
X comprises a nitric oxide releasing moiety.

4. The nitric oxide releasing polysiloxane of claim 3, wherein R¹ is a substituted or unsubstituted C₁-C₁₂ alkyl group and R² is a substituted or unsubstituted C₁-C₁₂ alkyl group.

5. (canceled)

6. (canceled)

7. (canceled)

8. The nitric oxide releasing polysiloxane of claim 1, wherein the nitric oxide releasing moiety comprises a S-nitrosothiol compound.

9. The nitric oxide releasing polysiloxane of claim 1, wherein the nitric oxide-donating moiety is a residue of S-nitroso-N-acetyl-penicillamine, S-nitroso-N-acetyl cysteine, S-nitroso-N-acetyl cysteamine, S-nitrosoglutathione, methyl S-nitrosothioglycolate, and a derivative thereof.

10. The nitric oxide releasing polysiloxane of claim 1, wherein the polysiloxane comprises a copolymer having a plurality of units of structure I and a plurality of units having the structure II 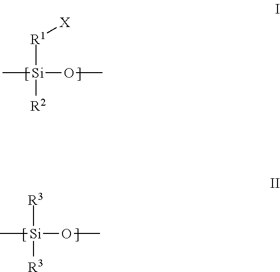
wherein R¹ is a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy;
R² is an alkyl group;
X comprises a nitric oxide releasing moiety; and
wherein R³ is an alkyl group.

11. (canceled)

12. (canceled)

13. The nitric oxide releasing polysiloxane of claim 1, wherein the nitric oxide releasing polysiloxane is produced the method comprising:
reacting a thiolactone with a polysiloxane comprising a polysiloxane backbone with one or more amino groups pendant to the polysiloxane backbone to produce a thiol-functionalized polysiloxane, and
nitrosating a thiol group on the thiol-functionalized polysiloxane to produce the nitric oxide releasing polysiloxane.

14-24. (canceled)

25. The nitric oxide releasing polysiloxane of claim 1, wherein the polysiloxane comprises one or more units having the structure IV 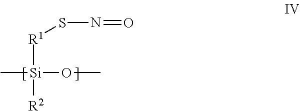
wherein R¹ is a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy; and
R² is an alkyl group.

26. The nitric oxide releasing polysiloxane of claim 25, wherein R¹ is a substituted or unsubstituted C₁-C₁₂ alkyl group.

27. (canceled)

28. The nitric oxide releasing polysiloxane of claim 25, wherein R² is a substituted or unsubstituted C₁-C₁₂ alkyl group.

29. (canceled)

30. The nitric oxide releasing polysiloxane of claim 1, wherein the nitric oxide releasing polysiloxane is produced the method comprising nitrosating a thiol group on a thiol-functionalized polysiloxane to produce the nitric oxide releasing polysiloxane.

31. The nitric oxide releasing polysiloxane of claim 31, wherein the polysiloxane backbone comprises a dialkyl polysiloxane.

32. The nitric oxide releasing polysiloxane of claim 30, wherein the thiol-functionalized polysiloxane comprises one more units having the structure V; 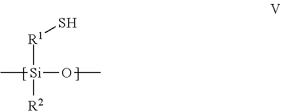
wherein R¹ is a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ herteroalkenyl, a substituted or unsubstituted C₁-C₂₀ alkoxy, or a substituted or unsubstituted C₁-C₂₀ heteroalkoxy; and R² is an alkyl group.

33. The nitric oxide releasing polysiloxane of claim 32, wherein R¹ is a substituted or unsubstituted C₁-C₁₂ alkyl group.

34. (canceled)

35. The nitric oxide releasing polysiloxane of claim 32, wherein R² is a substituted or unsubstituted C₁-C₁₂ alkyl group.

36. (canceled)

37. (canceled)

38. (canceled)

39. An article comprising at least one surface, wherein the at least one surface is coated with the nitric oxide releasing polysiloxane of claim 1.

40. An article comprising one or more components fabricated with the nitric oxide releasing polysiloxane of claim 1.

41-50. (canceled)

51. A method of preventing bacterial growth on a surface of an article, the method comprising applying the nitric oxide releasing polysiloxane of claim 1 to the surface.

52. A method of preventing biofilm formation on a surface of an article, the method comprising applying the composition of claim 1 to the surface.
